(19) United States
(12) Patent Application Publication
(10) Pub. No.: US 2025/0025691 A1
(43) Pub. Date: Jan. 23, 2025

(54) CROSS-APPENDAGE KINETIC STIMULATOR

(71) Applicant: The Q, LLC, Grand Junction, CO (US)

(72) Inventor: John Philip Adams, Grand Junction, CO (US)

(73) Assignee: The Q, LLC, Grand Junction, CO (US)

(21) Appl. No.: 18/909,837

(22) Filed: Oct. 8, 2024

Related U.S. Application Data
(63) Continuation of application No. PCT/US2023/065527, filed on Apr. 7, 2023.
(60) Provisional application No. 63/329,016, filed on Apr. 8, 2022.

(51) Int. Cl. A61N 1/36; A61N 1/02; A61N 1/04

(52) U.S. Cl. CPC A61N 1/36025; A61N 1/025; A61N 1/0456; A61N 1/36031; A61N 1/36034

(57) ABSTRACT

Systems and methods for neural synchronization via timed and controlled kinetic stimulation. The method involves stimulating a first limb located on one side of a median plane and transverse plane, while simultaneously stimulating a second limb located on the opposite side of the planes. Subsequently, a third and fourth limb are stimulated in alternating fashion, also located on opposing sides of the median and transverse planes. The method includes dynamically adjusting the synchronization of the stimulation pulses based on timing signals generated by a software-controlled algorithm. Additionally, synchronization is further calibrated by monitoring and recalculating neural activity distribution based on real-time patient data, ensuring precise coordination of stimulation pulses across all limbs.

### PRIORITY

This application is a continuation of PCT Application No. PCT/US2023/0065527, filed Apr. 7, 2023, which claims the priority benefit of U.S. Provisional Application No. 63/329,016, filed Apr. 8, 2022, which are both incorporated herein by reference in their entirety.

### BACKGROUND

This disclosure relates in general to brain therapy, including neurostimulation and cognitive enhancement.

Brain therapy has become increasingly important for treating neurological conditions, cognitive impairments, and mental health disorders. Traditional methods of brain stimulation, such as invasive deep brain stimulation, often involve surgical procedures that carry significant risks and require long recovery times. Non-invasive alternatives, including transcranial stimulation, typically rely on external devices with limited flexibility and functionality. As a result, there is a growing need for advanced, non-invasive brain therapy systems that offer greater versatility, real-time monitoring, and personalized treatment. These systems should provide safe, effective, and adaptive therapies while minimizing patient discomfort and the risks associated with conventional methods.

### SUMMARY

The disclosure describes a method for brain therapy. The method includes stimulating a first limb originating on a first side of a median plane and first side of a transverse plane while stimulating a second limb originating on a second, opposite side of the median plane and a second, opposite side of the transverse plane; and, subsequently. stimulating a third limb originating on the second side of the median plane and the first side of the transverse plane while stimulating a fourth limb originating on the first side of the median plane and the second side of the transverse plane.

A second method for brain therapy includes providing a first effector to a first limb originating on a first side of a median plane and first side of a transverse plane, providing a second effector to a second limb originating on a second, opposite side of the median plane and a second, opposite side of the transverse plane, providing a third effector to a third limb originating on the second side of the median plane and the first side of the transverse plane and providing a fourth effector to a fourth limb originating on the first side of the median plane and the second side of the transverse plane.

Further, the system includes first, second, third and fourth effectors each configured to stimulate a limb and a controller configured to activate the second effector while activating the first effector and subsequently activate the fourth effector while activating the third effector.

In one exemplary embodiment, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

One general aspect includes a method for providing brain therapy to maintain neural synchronization through timed and controlled stimulation pulses. The method also includes stimulating a first limb of a body located on a first side of a median plane and a first side of a transverse plane, while stimulating a second limb located on a second, opposite side of the median plane and a second, opposite side of the transverse plane. The method also includes subsequently stimulating a third limb located on a second side of the median plane and the first side of the transverse plane, while stimulating a fourth limb located on the first side of the median plane and the second side of the transverse plane.

The method also includes where the median plane is defined as a vertical plane that divides the body into left and right halves, and the transverse plane is defined as a horizontal plane that divides the body into upper and lower parts.

The method also includes dynamically adjusting synchronization of the stimulation pulses across the limbs based on timing signals generated by a software-controlled algorithm. The method also includes calibrating the synchronization of the stimulation pulses across the limbs by monitoring and recalculating a distribution of neural activity based on real-time patient data. Other embodiments of this aspect include corresponding computer systems, apparatus, and computer programs recorded on one or more computer storage devices, each configured to perform the actions of the methods.

Implementations may include one or more of the following features. The method where the synchronization timing of the stimulation pulses is dynamically adjusted within a range of 100 to 200 milliseconds, based on timing signals generated by the software-controlled algorithm. The method may include automatically adjusting a frequency and intensity of the stimulation pulses based on real-time system feedback from the software-controlled algorithm, while maintaining synchronization within a range of less than 200 milliseconds. The synchronization of the stimulation pulses occurs with a delay of less than 150 milliseconds, based on clock-based synchronization signals.

The method may include varying stimulation intensity between the first and second limbs, and the third and fourth limbs, with intensity modulation occurring in synchronization with the pulse timing based on the software-controlled algorithm. The stimulation pulses are delivered in predetermined waveform patterns selected from sinusoidal, square, or triangular, with a waveform type being dynamically selected by a control system based on system-specific feedback and patient-specific neural response profiles. The stimulation pulses are synchronized by distributing clock signals to a control unit and effectors and where the synchronization of the stimulation pulses is further adjusted based on real-time measurements of electrical impedance from electrodes attached to the body of a patient.

The method may include modulating intensity and frequency of the stimulation pulses in response to detected changes in heart rate of a patient. The method may include: dynamically adjusting, via a control system, a waveform type, intensity, and frequency of the stimulation pulses based on feedback signal received from a plurality of sensors sensing physiological parameters of a patient; and dynamically adjusting the synchronization and intensity of the stimulation pulses based on a feedback signal measured by a plurality of sensors. Implementations of the described techniques may include hardware, a method or process, or computer software on a computer-accessible medium.

One general aspect includes a system for providing brain therapy to maintain neural synchronization through timed and controlled stimulation pulses. The system also includes a plurality of transducers configured to be positioned on a first limb located on a first side of a median plane and a first side of a transverse plane, and a second limb located on a second, opposite side of the median plane and a second, opposite side of the transverse plane, as well as a third limb located on a second side of the median plane and the first side of the transverse plane, and a fourth limb located on the first side of the median plane and the second side of the transverse plane. The system also includes a control unit operatively connected to the plurality of transducers, the control unit configured to generate and deliver stimulation pulses to the transducers.

The system also includes a timing circuit or software-controlled algorithm in the control unit, configured to dynamically adjust the synchronization of the stimulation pulses across the limbs by generating timing signals. The system also includes a feedback processing unit operatively connected to sensors configured to monitor physiological data of a patient in real-time, the feedback processing unit configured to recalibrate and adjust the synchronization of the stimulation pulses based on a distribution of neural activity and patient-specific physiological data.

The system also includes where the control unit is configured to adjust synchronization of the stimulation pulses. Other embodiments of this aspect include corresponding computer systems, apparatus, and computer programs recorded on one or more computer storage devices, each configured to perform the actions of the methods.

Further, the system also includes a monitoring module configured to collect real-time physiological data from the patient during therapy, including neural response times, and adjust stimulation timing accordingly. The system also includes a cloud platform configured to securely store patient data and utilize machine learning algorithms to refine and optimize future therapy protocols. Other embodiments of this aspect include corresponding computer systems, apparatus, and computer programs recorded on one or more computer storage devices, each configured to perform the actions of the methods.

The summary above, as well as the following detailed description of illustrative embodiments, is better understood when read in conjunction with the appended drawings. For the purpose of illustrating the disclosure, example constructions are shown in the drawings. However, the disclosure is not limited to specific methods and instrumentalities disclosed herein. Moreover, those having ordinary skill in the art will understand that the drawings are not to scale. Wherever possible, like elements have been indicated by identical numbers.

### BRIEF DESCRIPTION OF THE FIGURES

Embodiments of the disclosure will now be described, by way of example only, with reference to the following diagrams wherein:

FIG. 1 illustrates a flow of an example method for improving brain function;

FIG. 2A illustrates an example method for improving brain function in a first phase;

FIG. 2B illustrates the example method of FIG. 2A in a second phase;

FIG. 3A illustrates an example system for improving brain function in a first treatment phase;

FIG. 3B illustrates the example system of FIG. 3A in a second treatment phase;

FIG. 3C illustrates an exemplary system for improving brain function with a plurality of effectors;

FIG. 4 schematically illustrates an example system for improving brain function;

FIG. 5 illustrates an example circuit diagram for disclosed systems for improving brain function;

FIG. 6A illustrates another example system for improving brain function in a first phase;

FIG. 6B illustrates the example system of FIG. 6A in a second phase;

FIG. 7A illustrates system architecture for generating kinetic stimulation using wired configuration;

FIG. 7B illustrates another system architecture for generating kinetic stimulation using wireless configuration;

FIG. 8 illustrates a system for brain therapy controlled by a central unit to maintain neural synchronization with real-time feedback;

FIG. 9 illustrates a detailed schematic diagram of effectors;

FIG. 10 illustrates a detailed schematic diagram of effectors with wirelessly connectivity;

FIG. 11 illustrates a pulse pattern used during brain therapy;

FIG. 12 illustrates synchronized delivery of two frequencies for brain therapy;

FIG. 13 illustrates synchronized delivery of two frequencies for brain therapy;

FIG. 14 illustrates a block diagram of apparatus for providing therapy or treating patients;

FIG. 15 illustrates a flowchart illustrating a method for testing the functionality of transducers in the stimulator system; and

FIG. 16 illustrated a flowchart demonstrating an exemplary method for synchronizing the stimulator system.

### DETAILED DESCRIPTION

The following detailed description illustrates embodiments of the disclosure and manners by which they can be implemented. Although the preferred mode of carrying out disclosed systems, apparatuses and methods has been described, those of ordinary skill in the art would recognize that other embodiments for carrying out or practicing disclosed systems, apparatuses and methods are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

The human brain demonstrates a frequent tendency to lose optimal function in one or both hemispheres (i.e., right and/or left brain). The reason this occurs can be hypothesized by demonstrating its correction and return to optimal function.

In addition, when in this suboptimal state, the human brain may become confused about the identity (frequency) of important substances, both inside and outside the human body. For optimal health, these substances need to be accurately identified and properly managed by the human organism. Examples of these substances can be nutrients, toxins, body tissues and fluids and chemicals such as hormones, neurotransmitters, etc. The brain also often does not properly process emotions and stress, creating a potentially unhealthy state, where these stresses are stored, negatively effecting the body.

One unhealthy brain condition known as Neurologic Disorganization may result in patients complaining of brain fog and/or reversing or switching words and numbers. A trained practitioner of Applied Kinesiology can diagnose Neurologic Disorganization by establishing a baseline muscular strength level of a major muscle such as the pectoralis clavicular and then retesting the strength of the muscle during left brain and right brain activities. Reduced strength of the muscle while the patient is performing a right brain activity such as humming indicates Neurologic Disorganization of the right side of the brain while reduced strength of the muscle while the patient is performing a left brain activity such as forward or backward counting indicates Neurologic Disorganization of the left side of the brain.

Known therapy for neurologic disorganization involves cross-pattern exercises such as lying prone and simultaneously raising and lowering a first leg and the arm on the opposite side of the mid-sagittal plane and then raising and lowering the other leg and the other arm. The exercise may be repeated for several minutes. The success rate of this type of therapy can be low and is prone to patient error in performing the exercises.

Embodiments of the disclosure provide electronic pulsing that assists the right and left sides of the human or animal brain to communicate more efficiently by providing a pulsing across the body midline.

Embodiments of the disclosure substantially eliminate, or at least partially address, problems in the prior art, enabling improved brain function without requiring a particular movement pattern by the patient.

Additionally, disclosed methods and systems optimize brain function, thus balancing right and left brain processing and communication. During this process the brain may “re-learn” the specific frequencies of nutrients, toxins, body organs, tissues, fluids, and more, with external exposure to the substances and/or their frequencies. Once the brain “re-learns” the correct frequencies of these substances, it no longer overreacts to or ignores these substances, but instead manages them appropriately. The brain may also process the internalized, unhealthy emotional stress that was suboptimally processed before.

Additional aspects, advantages, features and objects of the disclosure will be made apparent from the drawings and the detailed description of the illustrative embodiments construed in conjunction with the appended claims that follow. It will be appreciated that described features are susceptible to being combined in various combinations without departing from the scope of the disclosure as defined by the appended claims.

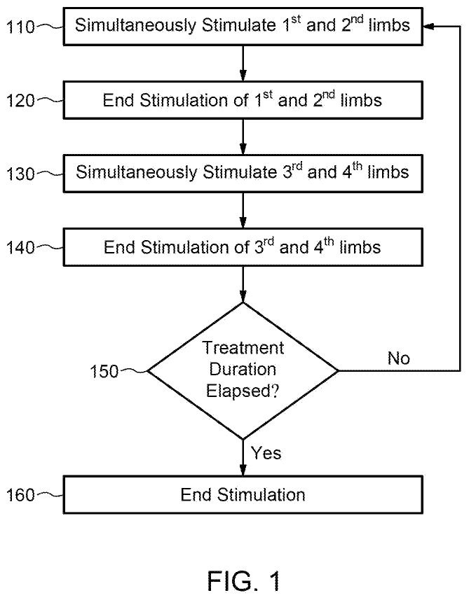

FIG. 1 illustrates a flow of an example method for brain therapy, improving brain function and/or treating brain injury. The method may be applied for treatment of bipedal organisms which are typically thought of as having sagittal planes or to quadrupedal organisms which are typically thought of as having median planes.

At 110, a first limb originating on a first side of a median or sagittal plane and first side of a transverse plane is stimulated while a second limb originating on a second, opposite side of the median or sagittal plane and a second, opposite side of the transverse plane is also stimulated. For example, a first effector, contact or probe is provided to the first limb, a second effector, contact or probe is provided to the second limb and the effectors are activated substantially simultaneously. The effectors may be provided so as to each encircle a portion of one of the limbs.

Further, the method as described in FIG. 1 involves stimulating limbs of a bipedal or quadrupedal organism to improve brain function, enhance neural synchronization, or treat brain injuries. The method is designed to account for the anatomical planes that divide the organism's body and guide the stimulation of specific limbs.

In this context, the median plane, also referred to as the median sagittal plane, is an anatomical term that refers to the vertical plane dividing the body into two equal left and right halves. For bipedal organisms, such as humans, the median plane runs longitudinally through the center of the body, extending from the head to the feet, and bisecting the body into symmetric left and right halves. For quadrupedal organisms, the median plane similarly divides the body along its central axis, separating the left and right halves of the body. The sagittal plane refers to any vertical plane parallel to the median plane that divides the body into left and right parts, though not necessarily symmetrically.

In addition, the transverse plane, also known as the horizontal plane, is an anatomical plane that divides the body into superior (upper) and inferior (lower) parts in bipedal organisms. This plane runs perpendicular to the median plane and the longitudinal axis of the body. In quadrupedal organisms, the transverse plane similarly divides the body into upper and lower parts, but it runs perpendicular to the body's long axis in a horizontal fashion.

In one exemplary embodiment of the present disclosure, the stimulation at step 110 is designed to occur based on these anatomical planes. Specifically, a first limb that originates on the first side of the median plane and the first side of the transverse plane is stimulated. This could involve, for example, stimulating the right hand of a human (located on the right side of the median plane and above the transverse plane) or the right forelimb of a quadrupedal animal. Simultaneously, a second limb that originates on the second, opposite side of the median plane and the second, opposite side of the transverse plane is also stimulated. For example, this could involve stimulating the left foot in a human (on the left side of the median plane and below the transverse plane) or the left hind limb in a quadrupedal animal.

This symmetrical stimulation across the anatomical planes is intended to engage both hemispheres of the brain and promote neural synchronization. The method leverages the concept of bilateral symmetry—where organisms have mirrored body parts on each side of the median plane—by stimulating corresponding limbs on opposite sides of the body. By alternating the stimulation between limbs on either side of these planes, the method seeks to enhance communication between the left and right hemispheres of the brain, which is critical for improving cognitive function and facilitating neural recovery following brain injuries.

For example, stimulating the right hand (on the first side of the median plane and above the transverse plane) and the left foot (on the second side of the median plane and below the transverse plane) simultaneously may help in treating conditions such as hemiplegia, where one side of the body is paralyzed due to brain injury. This stimulation method engages both the sensory and motor cortices in both hemispheres of the brain, potentially aiding in neural regeneration and the restoration of motor function.

In one exemplary embodiment of the present disclosure, by utilizing these anatomical planes as reference points, the method ensures that stimulation is evenly distributed across the body, which is particularly useful for treatments that require the synchronization of neural activity across both hemispheres of the brain. The method's flexibility allows it to be applied to various bipedal or quadrupedal organisms, providing a robust framework for treating a wide range of neurological conditions.

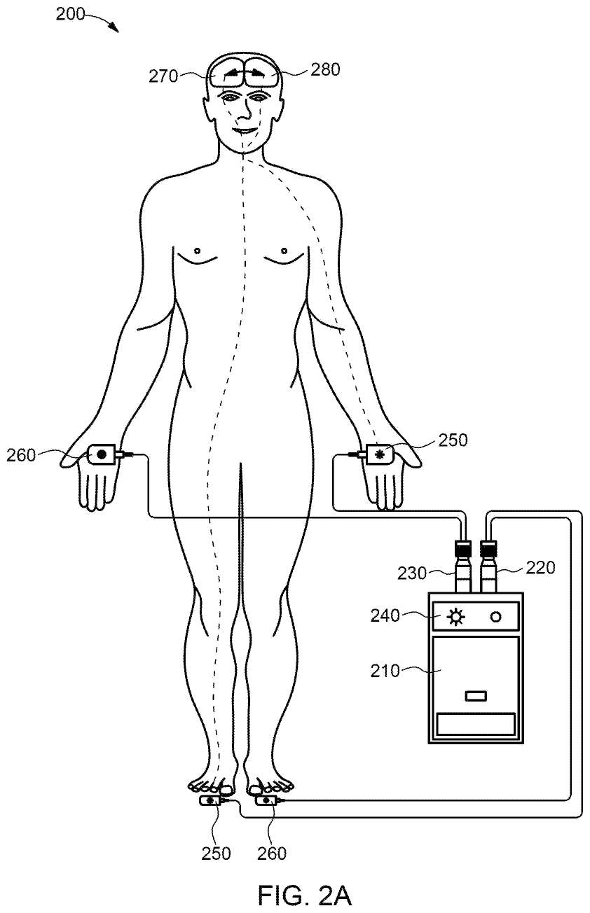

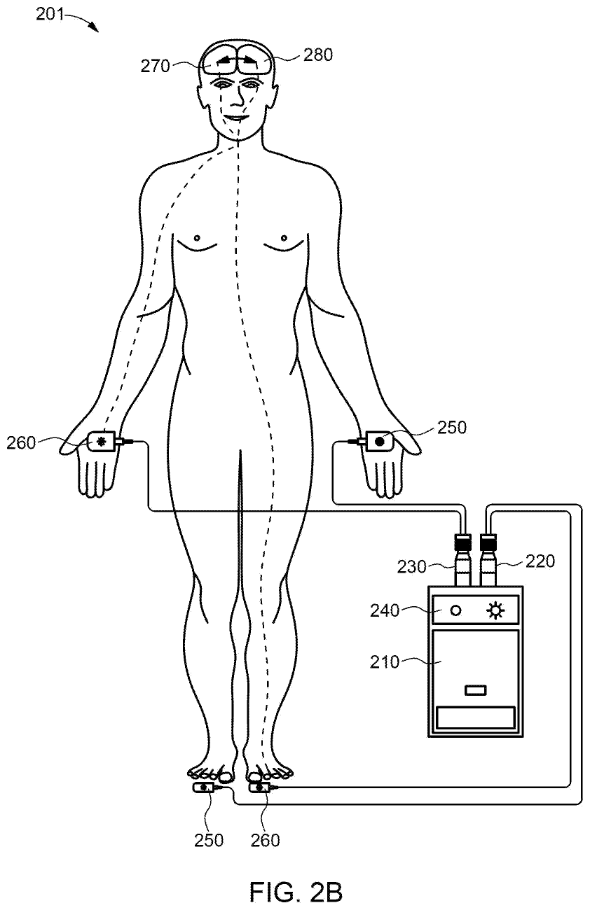

Referring to FIGS. 2A and 2B illustrates the example method of brain therapy of FIG. 1 in a first phase and second phase respectively. The present disclosure relates to a system 200 for brain therapy, as depicted in FIGS. 2A and 2B. The system (200) is designed to provide an electrical/kinetic stimulation to activate specific regions of the brain, particularly the left and right hemispheres, to improve cognitive and neurological function.

The system comprises a control unit (210), which is responsible for managing the electrical signals that are transmitted to the probes attached to the body of a patient by means of attachments (e.g. rubber band, clips etc.). The control unit (210) is equipped with connectors (220 and 230), which facilitate the attachment of probes (250 and 260). These probes (250 and 260) are positioned on specific parts of the patient's body, such as the hands or feet, to deliver electrical impulses to the brain via the body's neural pathways.

Further, in one exemplary embodiment of the present disclosure, the control module and indicators (240), located within the control unit, regulate the intensity and frequency of the electrical stimulation and provide feedback on the system's operational status. Visual indicators, such as LED lights, are used to signal the various states of the system, including power status and active therapy. The probes (250 and 260), connected via the control unit's connectors, are strategically positioned on the patient's hands or other targeted areas. These probes deliver electrical impulses that stimulate the left brain (280) and right brain (270), facilitating neural activation and plasticity.

Further, the therapy provided by system 200 is based on the principle of lateralized brain functions. The left brain (280) is commonly associated with logic, language, and analytical processing, whereas the right brain (270) is linked to creativity, emotion, and spatial reasoning. By stimulating both hemispheres simultaneously through the use of probes (250 and 260), the system aims to create a balanced activation of neural circuits, enhancing overall brain function.

In one exemplary embodiment, the system is employed for the rehabilitation of patients suffering from neurological conditions such as stroke, PTSD, OCD, ADD etc. The control unit (210) delivers electrical impulses through the probes to stimulate the affected hemisphere of the brain, promoting neural recovery and re-establishing lost functions. The intensity and frequency of the stimulation can be customized according to the patient's condition and progress, providing a tailored therapeutic experience.

In another exemplary embodiment, the system is used for cognitive enhancement in healthy individuals. By positioning the probes (250 and 260) on the hands during mental tasks, low-level electrical stimulation is applied to improve focus, problem-solving, and creativity. The system provides bilateral brain stimulation, ensuring that both hemispheres are actively engaged during cognitive tasks, thus optimizing brain performance.

In another exemplary embodiment, the system is adapted to eliminate the need for surgical intervention, making it a safer option for patients for treatment. Furthermore, the system's ability to provide bilateral stimulation ensures balanced activation of both brain hemispheres, a significant improvement over traditional one-sided stimulation methods. The system is also modular and customizable, allowing for adjustable stimulation settings and easy modification based on individual therapeutic needs. Additionally, its portability enables users to engage in therapy sessions in the comfort of their own homes or in clinical environments, thus improving patient compliance and convenience.

Furthermore, in one exemplary embodiment, stimulating the first limb while stimulating the second limb may be performed for a duration of between about 0.25 seconds and about 1.0 second. At 120, stimulation of the first and second limbs is ended. For example, first and second effectors may be substantially simultaneously deactivated.

At 130, a third limb originating on the second side of the median or sagittal plane and the first side of the transverse plane is stimulated while a fourth limb originating on the first side of the median or sagittal plane and the second side of the transverse plane is stimulated. For example, a third effector, contact or probe is provided to the third limb, a fourth effector, contact or probe is provided to the fourth limb and the effectors are activated substantially simultaneously. While illustrated as two separate actions, ending stimulation of the first and second limbs at 120 may, in practice, be contemporaneous with initiation of stimulating the third and fourth limbs at 130.

In one another exemplary embodiment, FIG. 2B also illustrates the example method of FIG. 1 in a second phase. Stimulating the third limb while stimulating the fourth limb may be performed for a duration of between about 0.25 seconds and about 1.0 second.

At 140, the stimulation of the third and fourth limbs is ended. For example, the third and fourth effectors may be substantially simultaneously deactivated. The activating and deactivating the first, second, third and fourth effectors may further include activating and deactivating with a controller. The plurality of effectors may be activated by wire or wirelessly.

At 150, it is determined whether the chosen treatment duration has elapsed. If the treatment duration has not elapsed, the method returns to repeating simultaneous stimulation of the first and second limbs at 110. As with actions 120 and 130, while illustrated as two separate actions, ending stimulation of the third and fourth limbs at 130 may, in practice, be contemporaneous with initiation of stimulating the first and second limbs at 110.

With substantially simultaneous, activation and/or deactivation of the various effectors, activation and/or deactivation may be sufficiently simultaneous that a patient being treated in accordance with the method would perceive the activation and/or deactivation to be simultaneous even if there is a small delay between the actions which would be detectable with instrumentation. Stimulating of the first, second, third and fourth limbs may be by stimulating with pulses, vibrations, electrical shock, negative-positive polarity switching, light and combinations thereof. Stimulating with light may be by activating and deactivating one or more bulbs or LEDs.

Repeating the stimulating the first limb while stimulating the second limb and subsequently repeating stimulating the third limb while stimulating the fourth limb may be performed for a duration of between about 1 and about 10 minutes. When the treatment duration has elapsed, stimulation of all limbs is ended at 160.

The actions described above are only illustrative and other alternatives can also be provided where one or more actions are added, one or more actions are removed, or one or more actions are provided in a different sequence without departing from the scope of the claims herein.

Disclosed methods for brain therapy improving brain function and/or treating brain injury may be performed by any of a variety of systems for brain therapy. The systems may be suitable for treatment of bipedal organisms with sagittal planes and to quadrupedal organisms with median planes.

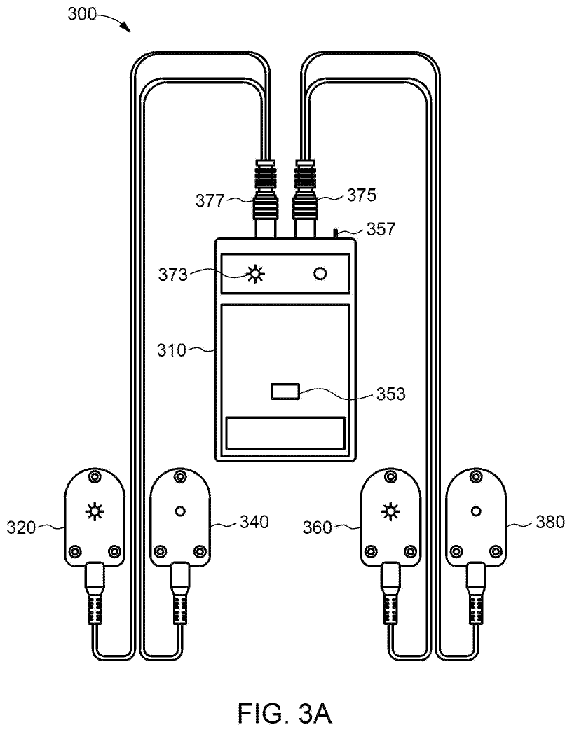

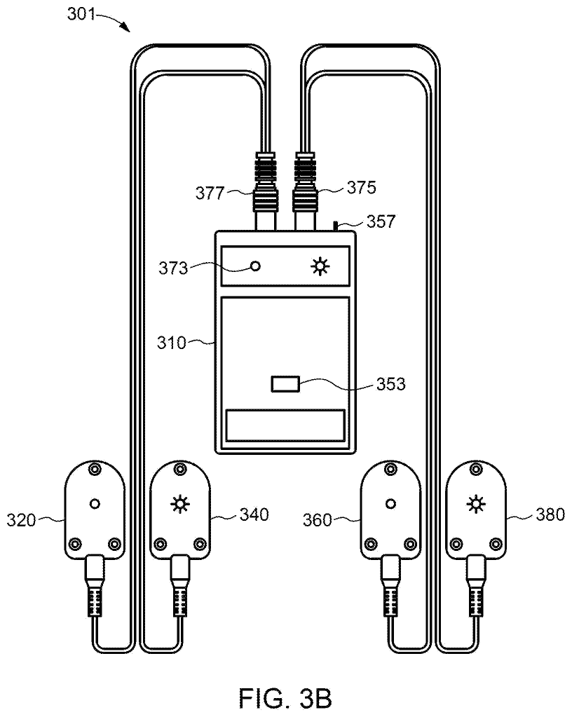

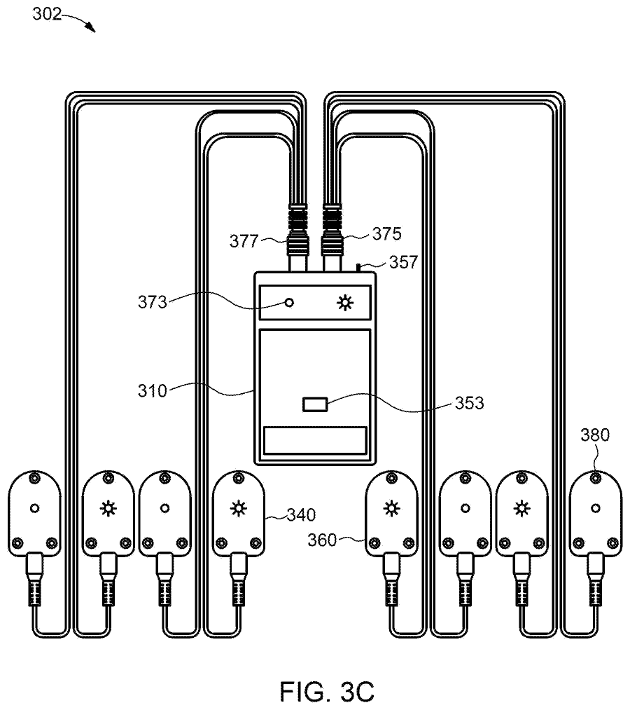

FIGS. 3A and 3B illustrates an example system 300 for brain therapy, improving brain function and/or treating brain injury in a first treatment phase. System 300 includes first 320, second 340, third 360 and fourth 380 effectors each configured to stimulate a limb.

First effector 320 is configured to stimulate a first limb originating in a first quadrant. For example, first effector 320 stimulates a limb originating on a first side of a median or sagittal plane and first side of a transverse plane.

Second effector 340 is configured to stimulate a second limb originating in a third quadrant. For example, second effector 340 is configured to stimulate a second limb originating on a second, opposite side of the median or sagittal plane and a second, opposite side of the transverse plane while the first effector is stimulating the first limb.

Third effector 360 is configured to stimulate a third limb originating in a second quadrant. For example, third effector 360 is configured to subsequently stimulate a third limb originating on the second side of the median or sagittal plane and the first side of the transverse plane.

Fourth effector 380 is configured to stimulate a fourth limb originating in a fourth quadrant. For example, fourth effector 380 is configured to stimulate a fourth limb originating on the first side of the median or sagittal plane and the second side of the transverse plane while the third effector is stimulating the third limb.

The first, second, third and fourth effectors may be configured to stimulate with pulses, vibrations, electrical shock and or negative/positive polarity.

The stimulating the first, second, third and fourth limbs may further include stimulating with light. In an example, first, second, third and fourth effectors include one or more bulbs or LEDs.

The first, second, third and fourth effectors may be configured to stimulate with vibrations. For example, the first, second, third and fourth effectors include one or more eccentric rotating masses and/or one or more linear resonant actuators which produce vibrations having a frequency of between 130 and 180 hz.

The first, second, third and fourth limbs may be configured to stimulate with electrical shock. In an example, the electrical shock is between 70 and 120 volts.

The first, second, third and fourth effectors may be configured to contact the limbs of a patient in any of a variety of ways suitable for properly stimulating the limbs for treatment. For example, each effector may be configured with a feature capable of encircling a portion of a limb, may be adhered to a patient's skin or may be provided as a subdermal or transdermal implant.

The first, second, third and fourth effectors are configured to stimulate the limbs by coordinated activation with a head unit and/or programmable controller.

Head unit 310 which may be an implementation of Head unit 400 and vice versa, is configured to activate second effector 340 while activating first effector 320 and subsequently, with the first and second effectors deactivated, activate fourth effector 380 while activating third effector 360.

A selector switch 353 enables selecting a treatment protocol from among a variety of treatment protocols. In an example, selector switch 353 is a three-position slider switch enabling selection of one of three programs stored on data memory 440 such as, for example, the 1-minute, 5-minute and 10-minute protocols described above.

A Pair of LEDs 373 are configured to illuminate in alternate order in synchronicity with switching activation of the effectors. Specifically, when one pair of effectors is activated, a first LED from the pair of LEDs 373 is illuminated and a second LED remains in OFF state. Conversely, when second pair of effectors are activated, the second LED is turned ON and the first LED goes to OFF state as shown in FIG. 3A whereas, in FIG. 3B, the states are reversed.

Stimulation jacks 375 and 377 output treatment signals to the remote effectors 320, 340, 360 and 380. In an example, the stimulation jacks are 3.5 mm female RCA jacks configured for operative coupling with lead cables for the effectors with 3.5 mm male RCA terminals.

A power switch 357 enables powering the system on and off. In an example, power switch 357 is a two-position toggle such as the Jameco 106067.

Disclosed systems for brain therapy were tested on 19 participants enrolled in a six-week clinical trial including 1 session per week for the first 4 weeks for a total of 76 sessions. Of the participants, 5 were men and 14 were women. Participant ages ranged from 23 to 74. The system included first, second, third and fourth effectors each configured to stimulate a limb and a controller configured to activate the second effector while activating the first effector and subsequently activate the fourth effector while activating the third effector.

Participants completed a written questionnaire including approximately 200 questions directed to brain health. Each participant was also required to perform a Basic Metabolic blood panel and a CBC blood panel and was manually muscle tested at the beginning and end of each session to determine whether the participant was in a state of neurologic disorganization.

The participants testing positive for neurologic disorganization were given a 10-minute session on the system, with effectors properly placed at hands and feet. They were retested to confirm the neurologic disorganization had resolved and that both brain hemispheres were negative.

All participants were then given the same instructions and asked to consider one or more of the following topics or emotions: are you trying not to think about something in your life?; are you trying to not be emotional about something in your life?; are you bothered by not knowing the outcome of something in your life?; or are you feeling frustration, resentment, despair, inefficiency, depletion, anguish, or hopelessness? The participants were asked to add as many personally stressful thoughts regarding the topic as possible, until they began to feel uncomfortable. A feeling of chest pressure was the most common physical manifestation. The participants were all tested for neurologic disorganization at this point. 56% of participants tested positive.

Each participant was then given a 20-minute session with the system. After the 20-minute session, the participants were asked to repeat the testing for neurologic disorganization. In 100% of instances of participants having neurologic disorganization following the topical or emotional consideration, treatment with the system corrected the neurologic disorganization.

During week 5 of the 6-week trial, a questionnaire was completed by the participants. 14 questions were asked to ascertain what benefits, if any, were experienced by the participants from use of the device. All participants preferred to continue treatment. Referring to FIG. 3C illustrates an exemplary embodiment of a system 302 for providing brain therapy. The system 302 includes a control unit 310 that is operatively connected to a plurality of effectors, designated as 340, 360, 380, through corresponding connection cables 375 and 377. In this exemplary embodiment, the system 302 comprises eight effectors, however, the system is not limited to this specific number of effectors. The number of effectors may vary based on the therapeutic requirements and the targeted areas of neural stimulation.

Further, the control unit 310 is configured to deliver synchronized stimulation pulses to each of the effectors 340, 360, 380, which are positioned at various locations on the patient's body to facilitate neural synchronization. In this regard, the effectors are configured to administer various forms of stimulation, including kinetic, electrical, or vibratory stimulation, depending on the therapy being applied. The placement of the effectors on the patient may include, but is not limited to, positions on the hands, feet, or other anatomical regions as necessary to target specific neural pathways associated with the therapy.

Furthermore, the control unit 310 includes various components essential for the operation and synchronization of the stimulation pulses, such as a waveform generator, phase relationship manager, and intensity controller, as described in connection with previous embodiments (e.g., FIGS. 3A and 3B). These components ensure that the stimulation pulses are delivered with precise timing, waveform characteristics, and intensity to optimize therapeutic efficacy. Additionally, the control unit 310 is equipped with visual indicators 373 that communicate the operational status of the system, as well as the connection status of each effector.

In one exemplary embodiment of the present disclosure, the system 302 is powered by an internal battery and is configured to provide synchronized stimulation pulses across the multiple effectors, ensuring that the stimulation is coordinated and aligned with the therapeutic goals. In this regard, by utilizing eight or more effectors the ability to stimulate multiple neural pathways simultaneously can be enhanced, thereby promoting a more comprehensive therapeutic impact. Further, the use of additional effectors allows the system to cover a greater area of the patient's body, thereby targeting multiple brain regions associated with different motor, sensory, or cognitive functions. This multi-limb stimulation is particularly advantageous for therapies directed at treating complex neurological conditions, such as stroke rehabilitation, where simultaneous activation of multiple neural pathways is necessary to achieve optimal therapeutic outcomes.

Moreover, while FIG. 3C depicts eight effectors, the system is not constrained to this configuration. The number of effectors (320, 340, 360, and 380) and respective connector ports (say 375 and 377 as shown in FIG. 3A) can be scaled up or down depending on the therapeutic needs of the patient. For instance, certain therapies may require fewer than eight effectors, while others may necessitate the use of ten or more effectors, depending on the specific condition being treated and the desired therapeutic effect. In particular, the synchronized operation of the effectors, controlled by the control unit 310, ensures that the stimulation pulses are delivered in a coordinated manner, thereby maintaining neural coherence across the patient's body.

In one another exemplary embodiment of the present disclosure, the system 302 is configured to provide synchronized brain therapy to a plurality of individuals simultaneously, thus enabling group-based therapy applications such as team-building exercises, sports coaching, and other similar activities that benefit from collective neural stimulation. In this embodiment, the system allows for multiple individuals to receive brain therapy concurrently, fostering coordinated mental engagement, enhanced focus, and improved group dynamics, making it particularly effective in environments that require teamwork and collaboration.

Moreover, when the system is implemented with wired connections, the device 310 includes a plurality of connecting ports (e.g., 373, 357, 377), which are configured to connect a plurality of effectors, each of which can be attached to a different individual. The device 310 may include 8, 16, 24, or more ports, enabling the connection of a corresponding number of effectors to facilitate simultaneous therapy for a group of individuals. In this regard, each group member is outfitted with one or more effectors, and the control unit 310 delivers synchronized stimulation pulses to all effectors concurrently. The number of connecting ports may vary depending on the specific therapeutic requirements and the size of the group, ensuring that the system can be scaled to accommodate small, medium, or large teams without limiting the number of participants.

In one another exemplary embodiment, when the system operates with wireless connections, the device 310 is capable of wirelessly communicating with a plurality of effectors, which can be distributed among multiple individuals. This wireless configuration allows the system to deliver synchronized stimulation to all participants without the need for physical connections. In such embodiments, the wireless system can support 10, 20, or more effectors, depending on the size of the group and the therapeutic objectives. Moreover, the wireless capability allows group members to receive therapy even if they are not physically co-located, thus offering significant advantages in situations where the individuals may be in different parts of a facility or even in remote locations, provided they remain within the range of the wireless network.

Furthermore, the system is also configured to dynamically adjust the stimulation parameters for each individual based on real-time feedback received from sensors regardless of whether the system is implemented with wired or wireless connections. This ensures that the therapy is tailored to each participant while maintaining overall synchronization across the group. The system's adaptability allows it to accommodate varying group sizes and therapeutic needs, making it highly flexible and customizable for a wide range of applications.

In particular, by synchronizing stimulation pulses across multiple participants, the system promotes enhanced group focus, coordination, and mental alignment. This is particularly advantageous in environments such as sports coaching, where synchronized neural engagement can lead to improved team performance, or in corporate settings, where team-building exercises can benefit from collective mental focus and enhanced cognitive function.

In the wired configuration, the device 310 provides scalability through the use of multiple connecting ports. The number of ports is not fixed and may be increased or decreased based on the specific therapeutic requirements and the size of the group being treated. In this regard, different versions of the stimulation device can also be used depending on the size of group and requirement of the therapy. For example, the system may be configured with 8, 16, or even 24 ports, allowing a large number of individuals to be connected and receive synchronized therapy.

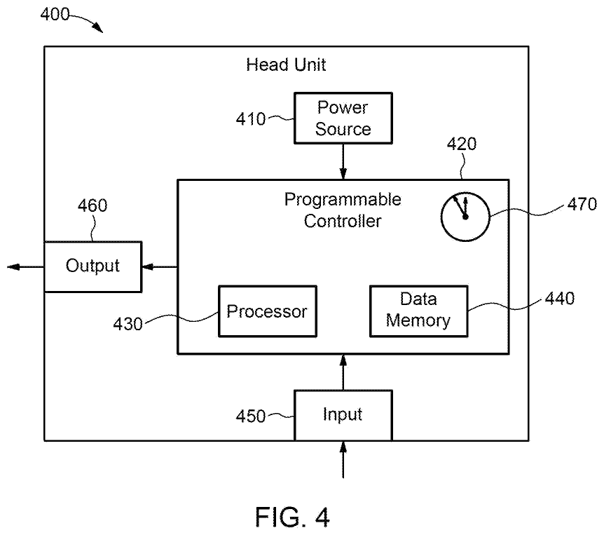

FIG. 4 schematically illustrates an example head unit 400 for controlling disclosed systems for improving brain function. Head unit 400 includes a programmable controller 420 which may have but is not limited to a data memory 440 and computing hardware such as a processor 430 which components may be implemented in a single system-on-a-chip. In an example, data memory 440 and computing hardware are implemented as a PIC16F616-I/SL system. A housing surrounds internal components of the head unit.

Programmable controller 420 is configured to activate the second effector while activating the first effector and subsequently, with the first and second effectors deactivated, activate the fourth effector while activating the third effector. Programmable controller 420 is configured to repeat these actions, switching between activating second and first effectors and activating fourth and third effectors, for a treatment duration.

Programmable controller 420 includes a timer 470 enabling counting a duration for switching between simultaneous activation of the first and second effectors and simultaneous activation of the third and fourth effectors. With the timer, programmable controller 420 may be configured to activate the second effector while activating the first effector for a duration of between about 0.25 seconds and about 1.0 second and configured to activate the fourth effector while activating the third effector for a duration of between about 0.25 seconds and about 1.0 second.

The timer 470 enables also counting duration of treatment. With the timer, programmable controller 420 may be configured to repeat stimulation of the first limb while stimulating the second limb and subsequently repeating stimulation of the third limb while stimulating the fourth limb for a duration of between about 1 and about 10 minutes.

Programming of controller 420 may enable providing any of a variety of treatment protocols. For example, data memory 440 stores a first, second and third programs for first, second and third treatment protocols. In a further example, when executed by processor 430, the first program is configured to alternate between the two stimulation types for a duration of 1 minute, the second program is configured to alternate between the two stimulation types for a duration of 5 minutes and the third program is configured to alternate between the two stimulation types for a duration of 10 minutes.

Head unit 400 also includes input devices 450 and output devices 460 supporting interactions with Head unit 400 and systems for improving brain function.

Input devices 450 include, for example, a selector switch which enables selecting a treatment protocol from among a variety of treatment protocols by selecting one of the programs stored on data memory 440.

Additionally or alternatively, input devices 450 may include a mouse or a joystick that is operable to receive inputs corresponding to clicking, pointing, and/or moving a pointer object on a graphical user interface. Input devices 450 may also include a keyboard that is operable to receive inputs corresponding to pushing certain buttons on the keyboard. Further, input devices 450 may also include a microphone for receiving an audio input from the user.

Examples of output devices 460 include indicators such as LEDs, audio speakers for producing sounds and display screens for presenting graphical images to a user of Head unit 400. In a further example, a provided display screen may be a touch-sensitive display screen that is operable to receive tactile inputs from the user. These tactile inputs may, for example, include clicking, tapping, pointing, moving, pressing and/or swiping with a finger or a touch-sensitive object like a pen.

Outputs devices 460 further include several stimulation jacks to output treatment signals to the remote effectors. Controller 420 may be configured to activate the first, second, third and fourth effectors by wire or wirelessly depending on the type of stimulation jacks provided.

Head unit 400 also includes a power source 410 for supplying electrical power to the various components of Head unit 400. Power source 410 may, for example, include a rechargeable battery.

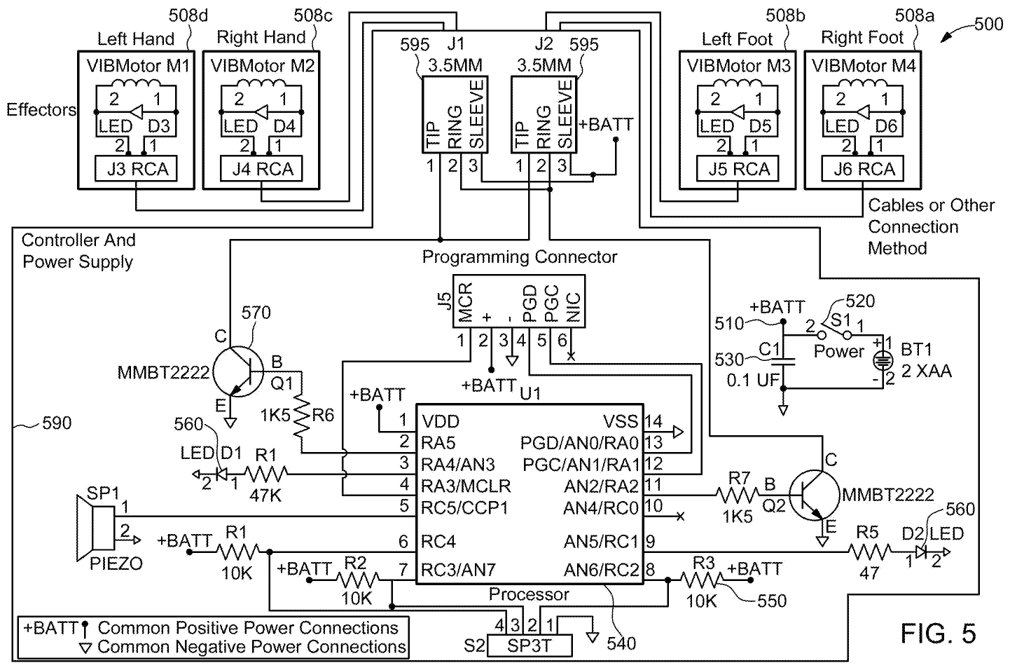

FIG. 5 illustrates an example circuit diagram for disclosed systems for improving brain function according to an embodiment of the present disclosure.

The present disclosure introduces a system for improving brain function through targeted electrical and kinetic stimulation, utilizing various interconnected components to deliver precise and effective therapy.

The system is powered by a portable battery (510), designed to supply the required electrical energy to all components. The battery can be a set of AA batteries or a rechargeable lithium-ion cell. This battery is for ensuring the device remains operational and portable, facilitating therapy sessions across different settings. The power flows from the battery to the rest of the circuit, enabling all downstream components to function.

Further, the system includes a power switch (520) positioned between the battery (510) and the rest of the circuit. This switch allows the user to control the flow of current to the device, providing a convenient means to turn the system ON or OFF. When the switch is activated, current flows from the battery to the other components, triggering the operation of the therapeutic processes.

Once the system is turned ON, the processor (540) functions as the control unit, controlling the device's overall operation. The processor (540) receives user inputs and executes pre-programmed therapy routines by controlling the flow of electrical and vibratory tapping signals. The processor (540) sends control signals to various outputs, including the vibrators (580) and piezo buzzer (590), based on the therapy requirements. Additionally, the processor (540) manages real-time adjustments by communicating with other components, such as the monitoring module (not shown), ensuring that the therapy remains adaptive and responsive to the patient's needs.

Further, various resistors (550) are deployed throughout the circuit, serving to regulate current flow to the components. They are essential for ensuring that key elements like the LEDs (560) and vibrators (580) operate within safe parameters. For instance, resistors are connected in series with the LEDs to prevent overdriving.

Furthermore, the LEDs (560) provides visual feedback to the user, indicating system status and functionality. The LED (560) is connected to the processor (540) and can be programmed to illuminate or blink in specific patterns, corresponding to different operational states such as therapy initiation, completion, or error conditions. This provides real-time information to both users and clinicians regarding the device's current mode of operation.

In one exemplary embodiment, the system 500 incorporates multiple vibrators 580 (580a, 580b, 580c, 580d), strategically placed on the patient's body to deliver kinetic stimulation. These vibrators 580 are driven by signals from the processor (540) via the transistor (570), which ensures they receive the appropriate current to operate effectively. The vibrators 580 can be adjusted individually, providing targeted stimulation to different parts of the body, such as the hands, feet, or scalp. This flexibility allows the system to tailor the therapy to the specific needs of each patient.

In addition, a piezo buzzer (590) is provided as another output component controlled by the processor (540) and powered via the transistor (570). It provides auditory feedback to the user, emitting sound cues that signal a start or end of a therapy session, as well as other operational statuses. The piezo buzzer (590) complements the vibratory tapping and visual feedback, creating a multi-sensory experience that enhances the efficacy of the therapy.

Further, the system 500 is equipped with modular connectors (595), such as 3.5 mm or RCA jacks, that facilitate easy attachment of peripheral devices, including vibrators and other stimulation components. These connectors (595) allow the system 500 to be customized based on the patient's specific therapy requirements. For instance, different types of vibrators 580 can be plugged in, or additional sensors can be integrated for advanced monitoring, enhancing the system's versatility.

In one exemplary embodiment, each of these components are connected wired/wirelessly to create a therapeutic system. The battery (510) powers the system, with the power switch (520) controlling activation. The capacitor (530) ensures smooth operation, allowing the processor (540) to effectively manage and direct therapy. Resistors (550) protect sensitive components like the LED (560), while the transistor (570) amplifies control signals, driving the vibrators (580) and Piezo buzzer (590). The connectors (595) provide modularity, making the system adaptable for various applications.

In one exemplary embodiment, once the power switch (520) is flipped ON, electrical current flows through the circuit, enabling all components to become active. A capacitor (530) immediately comes into play, smoothing out any voltage fluctuations that may occur as the device powers on. This ensures a stable flow of current, protecting sensitive components such as the processor (540). The processor (540) serves as the central control unit or the “brain” of the system, executing pre-programmed therapy routines. Further, the processor (540) is configured to control when and how the stimulation occurs, whether it's electrical impulses or vibratory tapping signals, based on user-defined settings or sensor feedback. When a therapy session begins, the processor (540) is configured to send control signals to the transistor (570). The transistor (570) acts as a gatekeeper, amplifying these control signals to drive the larger current loads necessary to operate the vibrators (580) and the Piezo buzzer (590).

For example, during a therapy session designed to stimulate cognitive function, the processor (540) might send varying pulse signals to the vibrators (580a, 580b, 580c, and 580d), strategically placed on the user's body, such as their hands, feet, or head. These vibrations provide tactile stimulation, to enhance neural activity and promote brain plasticity. The vibrators (580a, 580b, 580c, and 580d) are controlled to ensure intended timing and intensity, providing a stimulating experience without causing discomfort.

Meanwhile, the LED (560), linked directly to the processor, may serve as a status indicator, blinking in different patterns to signal various stages of the therapy. For example, it could pulse slowly during the relaxation phase or blink rapidly to indicate cognitive stimulation is in progress. This visual cue adds an additional layer of feedback for the user.

In one exemplary embodiment, the Piezo buzzer (590) is activated by the processor (540) to deliver auditory cues-such as beeps-signaling the start or end of a session, or to provide real-time feedback on the session's progress. This multi-sensory feedback—vibratory tapping, visual, and auditory—keep the user engaged, making the therapy more effective and immersive.

All the devices are connected via modular connectors (595), which provide flexibility in how the system is used. For example, different vibrators or other types of sensors can be plugged in based on the therapy being applied. These connectors make the system highly customizable, allowing the user or healthcare provider to adapt the device for various therapeutic needs.

In one another exemplary embodiment, a patient wears the system's vibratory tapping devices on their hands and feet, where the processor delivers carefully timed electrical impulses. The system is configured to begin with low-intensity vibrations to stimulate neural pathways and gradually increase intensity as therapy progresses. The transistor (570) ensures that each vibrator receives the proper current for the level of stimulation needed. As the session progresses, the piezo buzzer (590) signals the transition between different phases of the therapy, while the LED (560) provides visual confirmation that the system is functioning as expected.

In this scenario, the patient's brain receives the necessary stimulation to enhance neuroplasticity, facilitating the recovery of motor functions or cognitive abilities lost due to any disease.

FIGS. 4 and 5 are merely examples, which are not intended to unduly limit the scope of the claims herein. It is to be understood that the specific designation for Head unit 400 is provided as an example and is not to be construed as limiting Head unit 400 to specific numbers, types, or arrangements of modules and/or components. A person skilled in the art will recognize many variations, alternatives, and modifications of embodiments of the present disclosure.

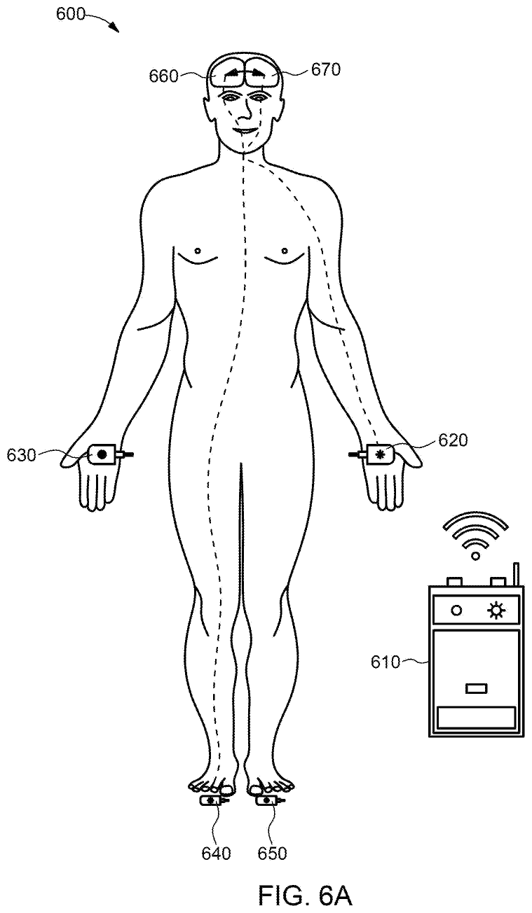

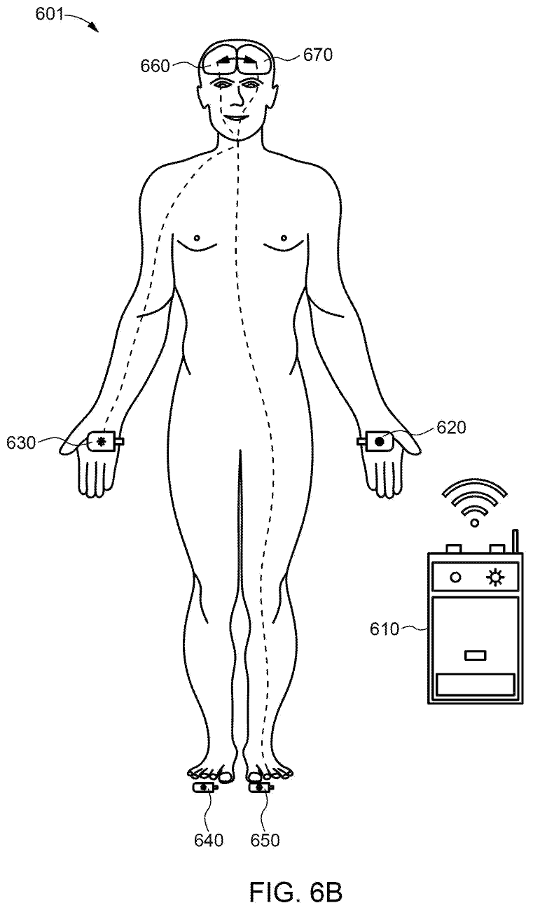

As illustrated, FIGS. 6A and 6B relates to a system for brain therapy, where electrical stimulation is delivered wirelessly to target regions of the brain to improve cognitive and neurological function. Similar to the embodiment described in FIGS. 2A and 2B, this system (600) comprises a control unit (610) and a plurality of probes (620, 630, 640, and 650), but the key difference lies in the wireless nature of the connection between the control unit and the probes.

Specifically, the control unit (610) communicates with the probes (620, 630, 640, and 650) via wireless transmission, eliminating the need for physical connectors. The control unit (610) manages the transmission of electrical stimulation signals wirelessly through built-in wireless transceivers. These transceivers establish a secure connection with the wireless receivers embedded within the probes (620, 630, 640, and 650), which are positioned on the patient's body to deliver the electrical impulses. The wireless communication is established using protocols such as Bluetooth, radio frequency (RF), near-field communication (NFC), or other appropriate wireless technologies, ensuring seamless and uninterrupted signal transmission.

The control module and indicators (640—not shown) located within the control unit (610) are responsible for regulating the intensity, frequency, and duration of the stimulation signals. The control unit (610) processes inputs from the user or clinician, adjusts the stimulation parameters, and wirelessly transmits the corresponding control signals to the probes (620, 630, 640, and 650). These probes, once positioned on the body-such as on the hands or feet-receive the wireless signals and convert them into electrical impulses. The stimulation delivered through the probes is specifically targeted to activate the left brain (670) and right brain (660), engaging neural pathways through non-invasive electrical impulses.

Once powered, the control unit (610) initiates a pairing process with the probes (620, 630, 640, and 650). The unit's transceiver searches for the receivers in the probes (620, 630, 640, and 650) and establishes a secure, encrypted connection. Once connected, the control unit (610) begins transmitting stimulation signals wirelessly. These signals instruct the probes (620, 630, 640, and 650) on how to adjust the intensity and frequency of the stimulation, ensuring precise and safe delivery of therapy.

In one exemplary embodiment, the probes (620, 630, 640, and 650) contain their own integrated circuits (ICs) and power sources, such as small rechargeable batteries, which enable them to function independently while converting wireless signals into electrical pulses that stimulate targeted areas of the body.

In one another exemplary embodiment, the control unit (610) continuously monitors the connection status with the probes (620, 630, 640, and 650), allowing for real-time adjustments of the therapy. Any feedback from the probes (620, 630, 640, and 650) is relayed back to the control unit, which can alter the stimulation parameters dynamically to ensure optimal therapeutic outcomes. The indicators (640—not shown herein) on the control unit provide visual cues to the user, signaling whether the system 600 is properly connected, actively delivering stimulation, or in need of attention due to a loss of connection or other system status.

The wireless connection between the control unit (610) and the probes (620, 630, 640, and 650) is initiated when the system is powered on. The control unit's wireless transceiver searches for and pairs with the wireless receivers embedded in the probes (620, 630, 640, and 650). The system 600 employs a secure, encrypted communication protocol, such as Bluetooth or RF, to ensure accurate and interference-free transmission. Once paired, the control unit (610) transmits alternate stimulation signals wirelessly, allowing the probes (620, 630, 640, and 650) to deliver the appropriate electrical impulses based on the therapy session's programmed parameters.

In one exemplary embodiment, the system (600) is employed in neuro-rehabilitation for patients recovering from conditions such as stroke. The probes (620, 630, 640, and 650), placed on the hands or feet, deliver targeted stimulation wirelessly, focusing on reactivating neural pathways in the affected hemisphere of the brain, specifically the left brain (670) or right brain (660). The wireless design of the system allows for greater mobility and comfort, enabling patients to move freely during therapy sessions without the restrictions imposed by wired systems. This is particularly advantageous for patients with mobility impairments, as it eliminates the risk of tripping or becoming entangled in wires during therapy.

In another embodiment, the wireless system is used for cognitive enhancement in healthy individuals. The control unit (610) pairs with discreet, portable probes (620, 630, 640, and 650) placed on the body, allowing users to receive low-level brain stimulation while performing everyday tasks.

In one exemplary embodiment, the wireless connection allows for continuous, real-time communication between the control unit and the probes. This enables dynamic adjustments to the stimulation parameters during therapy, ensuring that the therapy is always safe, effective, and tailored to the patient's needs. The feedback loop between the probes and the control unit ensures optimal performance and adaptability during treatment.

In one exemplary embodiment, the plurality of probes may be activated by wire or wirelessly.

In one another exemplary embodiment, the controller may be configured to activate the first, second, third and fourth probes/contacts by wire as shown by way of example in FIGS. 2A and 2B or wirelessly as shown by way of example in FIGS. 6A and 6B.

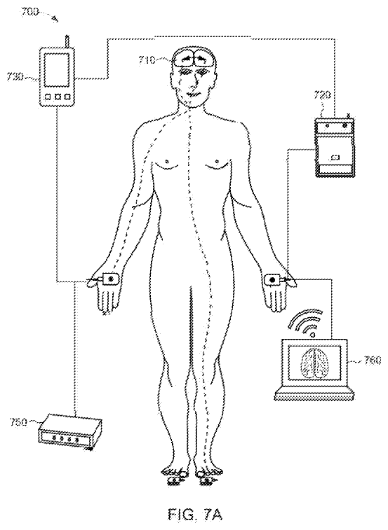

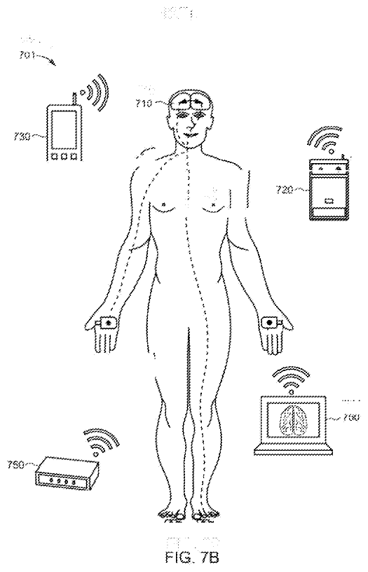

Referring to FIG. 7A and FIG. 7B, illustrates two distinct embodiments of the brain therapy system, configured for both wired and wireless operation. In FIG. 7A, the system is shown in its wired configuration, wherein the control unit 720 is operatively connected to a plurality of effectors via physical cables. On the other hand, as illustrated in FIG. 7B, the system is shown in a wireless configuration, wherein the control unit 720 communicates with the effectors wirelessly, eliminating the need for physical connections.

Both configurations are designed to deliver stimulation patterns in accordance with the details described in FIG. 6A and FIG. 6B, involving alternating sequences of stimulation pulses delivered to the limbs, with programmable delay intervals or without intervals as needed to maintain neural synchronization and optimize therapeutic outcomes. These embodiments provide versatility in system deployment, offering both the robustness of wired connectivity and the convenience of wireless operation, depending on the therapeutic requirements.

Specifically, FIG. 7A illustrates an example system (700) for brain therapy that delivers electrical or kinetic stimulation to targeted regions of the brain through a non-invasive, interconnected framework of devices. The system is designed to facilitate brain therapy for improving cognitive function, neuro-rehabilitation, or cognitive enhancement by stimulating neural pathways. The system (700) includes several components that work together to deliver targeted stimulation: patient (710), patient interface (730), control module (720), monitoring module (760), user interface (770), power supply (750), and control unit (740).

Specifically, the system (700) is designed for use on a patient (710), who receives the electrical or kinetic stimulation aimed at activating specific brain regions. The stimulation is delivered through non-invasive means, such as electrodes or vibratory tapping devices attached to the patient's body.

The system (700) includes a control module (720) that is configured to regulate the stimulation signals sent to a patient interface (730). This module processes inputs from the user interface (770) and the monitoring module (760) to determine the optimal intensity, frequency, and duration of the stimulation. It ensures that the patient interface (730) receives the appropriate stimulation signals based on the specific therapy regimen and makes any necessary adjustments dynamically during the session.

The system (700) further includes the patient interface (730) that serves as the medium for delivering the electrical or kinetic stimulation or checking the real time progress in health conditions due to therapy. The patient interface (730) includes electrodes or vibratory tapping devices placed on the patient's body, typically on the hands, feet, or other target areas, to stimulate corresponding brain regions via neural pathways. The patient interface receives commands from the control module (720) to ensure precise and effective delivery of the stimulation.

Additionally, the patient interface (730) may incorporate sensors that gather data on the patient's response to the stimulation, relaying that information back to the control module for real-time adjustments.

Further, the system 700 includes a monitoring module (760) that is configured to collect real-time physiological data from the patient (710) during the therapy session. This data may include metrics such as brainwave activity, heart rate, and other relevant parameters that help assess the effectiveness of the therapy.

The monitoring module continuously communicates with the control module (720), allowing the system to adjust stimulation in response to the patient's real-time feedback, thereby optimizing the therapeutic outcome.

The system further includes the user interface (770) that provides an interface through which a therapist or practitioner can manage and control the therapy session. Displayed on a laptop or mobile device, the user interface allows for adjustments to the therapy settings-such as altering the stimulation intensity or adjusting the duration of the session. The user interface also displays real-time data from the monitoring module (760), giving the user insights into the patient's progress and the system's performance.

Furthermore, the power supply (750) provides the necessary energy to operate the system components, including the control module (720), patient interface (730), and control unit (740). The power supply may consist of rechargeable batteries or direct power sources depending on the application, ensuring the system remains operational and reliable throughout therapy sessions.

The control unit (740) acts as the central hub that integrates all of the system's components. It manages communication between the patient interface (730), control module (720), monitoring module (760), and user interface (770). The control unit ensures that the correct stimulation parameters are relayed to the patient interface and that the therapy is adjusted dynamically in real-time based on feedback from the monitoring module.

In one exemplary embodiment of the disclosure, the patient interface (730) delivers kinetic stimulation to body regions connected to the brain's frontal lobe, stimulating focus and cognitive function. The system is controlled remotely via the user interface (770), allowing users to engage in cognitive tasks while receiving brain stimulation. The monitoring module (760) provides feedback on brain activity, ensuring optimal performance during cognitive training sessions.

Referring to FIG. 7B illustrates another system architecture for generating kinetic stimulation according to another embodiment of the present disclosure. Specifically, FIG. 7B illustrates a system similar to FIG. 7A in terms of architecture. However, all components are interconnected wirelessly, enhancing the system's flexibility and portability.

In this embodiment, patient data is securely transmitted and stored on a cloud platform with the consent of the patient, where it can be used to train machine learning algorithms for optimizing therapy outcomes. The system ensures that data security and privacy are maintained, offering a safe and efficient method for real-time brain therapy while leveraging advanced data analytics.

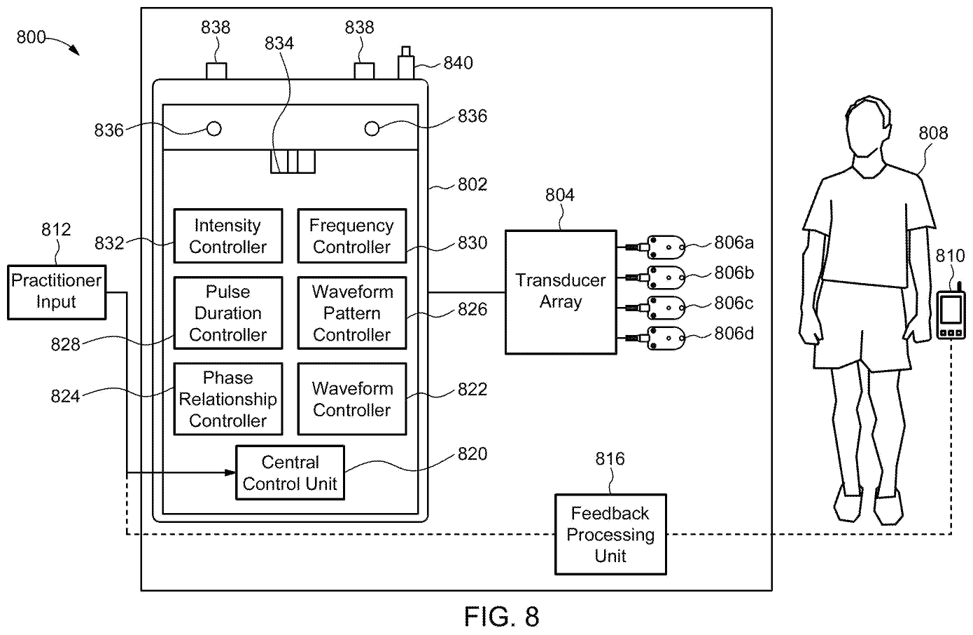

Referring to FIG. 8 illustrates a system 800 for brain therapy controlled by a central unit to maintain neural synchronization with real-time feedback according to an embodiment of the present disclosure. The system 800 is operable to maintain neural synchronization through controlled stimulation pulses, which are dynamically adjusted based on real-time feedback. The system 800 includes a control unit 802 that is operatively connected to a transducer array 804 either through a wired or wireless connection. The transducer array 804 is comprised of a plurality of effectors 806 (a, b, c, d) that are configured to be attached to a patient's body 808 for delivering therapeutic stimulation. The stimulation may be in the form of electrical or kinetic stimulation, depending on the selected treatment modality.

Further, a feedback device 810 (a sensory device for automatic feedback or a user interface for manual feedback) is positioned with the patient 808 to receive physiological feedback during the course of the therapy. The feedback device 810 is designed to collect patient-specific data, including, but not limited to, neural response, muscle activity, and skin conductivity. Once the feedback data is collected, the feedback device 810 communicates with a feedback processing unit 816, which is operatively configured to process the incoming feedback by filtering out noise and irrelevant data, thus ensuring that only clean, actionable data is transmitted to the control unit 802. This processed feedback allows the system to dynamically adjust the stimulation parameters in real time, optimizing the therapeutic outcomes for the patient.

In one exemplary embodiment, the control unit 802 is additionally configured to receive input from a practitioner 812, either directly or indirectly, allowing the therapy to be customized further based on clinical decisions. The control unit 802 houses a plurality of components necessary for controlling the stimulation parameters, including a central control unit 820, a waveform generator 822, a phase relationship controller 824 (PR controller), a waveform pattern controller 826, a pulse duration controller 828, a frequency controller 830, and an intensity controller 832. Each of these components are configured to adjust respective aspects of the stimulation, such as waveform shape, timing, duration, frequency, and intensity, thereby enabling highly tailored therapy sessions that respond dynamically to the patient's condition.

Moreover, the control unit 802 includes a three-program slider switch 834, which provides selectable pre-programmed therapy modes, and a pair of LED indicators 836 to visually communicate the operational status of the system. The unit is equipped with a pair of 3.5 mm female RCA jacks 838, allowing for external device connections, and is powered by a rechargeable battery, though it can be connected to a charging cable for continuous operation during extended therapy sessions. The system's operational state is controlled via an ON/OFF switch 840.

Further, the system is not limited to the use of a ON/OFF switch 840 (e.g. toggle switch) for turning the device ON and OFF. While the switch 840 is depicted as one possible method of controlling power, the system may be equipped with various alternative power control mechanisms, including, but not limited to, touch sensors, voice-activated controls, or motion detection systems. These alternative power control mechanisms may be integrated into the device to enable activation or deactivation through a user interface, mobile application, or other remote controls.

In this regard, this flexibility in power control allows the device to be tailored to different use environments and user preferences. For example, in a sterile medical environment, touch-sensitive or proximity-based controls may offer a more hygienic solution than traditional physical switches. Voice-activated commands or software-based control mechanisms can further enhance ease of use, allowing the device to be operated hands-free or through remote means, thus improving overall user experience and facilitating integration with other systems. Furthermore, incorporating advanced control mechanisms such as voice activation or software-based management may contribute to greater operational efficiency, energy conservation, and system integration in diverse applications. In addition, the power control mechanism of the device is not limited to the toggle switch 840 as shown in FIG. 8. Further, any suitable means of powering the device, whether mechanical, electronic, or software-based, may be employed without departing from the scope of the present invention.

In one another exemplary embodiment, the brain therapy is achieved through the synchronization of stimulation pulses and the incorporation of real-time feedback, which together maintain neural coherence and optimize the effectiveness of the therapy. The synchronization of stimulation pulses across the effectors 806 is essential for ensuring that the neural pathways are stimulated in a coordinated manner, which is vital for the therapeutic efficacy of the brain therapy. Additionally, the real-time feedback allows the system 800 to adjust stimulation parameters dynamically based on the patient's physiological responses, thus delivering a more personalized and effective treatment.

In one exemplary embodiment of the present disclosure, the timer 834 is configured to define a specific duration for the brain therapy session, with timing intervals adjustable based on the application, the subject's condition, and the therapeutic objectives. The timer 834 may be set for a range of time extending from as short as one minute to as long as two hours, or for even longer durations as necessary for a particular treatment.

In addition, the timer 834 is operatively connected to the control system, that is configured for the delivery of stimulation pulses through the transducers 806a, 806b, 806c, and 806d. In this regard, the therapist may configure the timer to ensure that the therapy session is conducted for an optimal period, tailored to the patient's condition. For example, in neuro-rehabilitation treatments, the timer may be set for extended durations to promote prolonged neural engagement, whereas in cognitive enhancement or relaxation therapies, shorter time frames may be more appropriate.

Furthermore, it should be expressly noted that the timing intervals mentioned herein, such as the range from one minute to two hours, are merely exemplary and not limiting. The system is fully capable of supporting a wide array of programmable time intervals, and any suitable timing configuration may be selected by the practitioner based on the specific therapeutic application. The flexibility of the timer 834 allows for both shorter and longer therapy sessions, as needed, ensuring that the system is adaptable to the individual needs of each patient and therapy. The broad timing range provided is intended to accommodate diverse therapeutic regimens without departing from the scope of the present invention, and the duration of the active stimulation phase may be customized without limitation to the specific examples provided herein.

In one exemplary embodiment of the present disclosure, synchronization is achieved through a central control unit equipped with timing circuits that regulate the activation of each transducer. The central control unit coordinates the timing of stimulation pulses across all transducers, ensuring that they operate in unison or in a controlled alternating sequence. Based on therapy parameters, the control unit generates synchronized timing signals, allowing each transducer to deliver kinetic stimulation precisely at the required intervals.

One another exemplary embodiment of the present disclosure involves the use of phase-locked loops (PLLs), which ensure that all transducers maintain a consistent phase relationship by locking onto a reference signal generated by the central control unit. This approach is particularly useful for maintaining precise phase alignment when consistent timing is critical to the therapeutic outcome.

Further, wireless communication protocols with timestamps are another method for achieving synchronization, especially in systems where the transducers operate wirelessly. Each transducer receives timestamped signals that dictate when it should activate, and based on these timestamps, each transducer synchronizes its operation with the central control unit, compensating for any propagation delays in the wireless signal.

Furthermore, real-time feedback loops offer another method for maintaining synchronization. Sensors monitor the output of the transducers during therapy, providing real-time data on the timing and intensity of the kinetic stimulation. Based on this feedback, the control unit dynamically adjusts subsequent pulses to ensure synchronization across the system.

In one exemplary embodiment of the present disclosure, closed-loop synchronization systems use patient feedback to regulate synchronization. A plurality of sensors attached to the patient continuously monitor physiological responses, such as heart rate, muscle tension, or neural activity, and based on this feedback, the system adjusts the timing of the stimulation pulses to maintain synchronized operation.

In one another exemplary embodiment of the present disclosure, software-controlled synchronization algorithms embedded in the control unit further assist in computing the optimal timing intervals required for synchronized operation.

Additionally, synchronized clock signals can be distributed to each transducer, ensuring that all transducers follow a common timing source. Based on these synchronized clock signals, the transducers can be triggered simultaneously or in a predetermined sequence, ensuring that the therapy remains consistent across multiple stimulation points. Latency compensation mechanisms are used in wireless systems to account for signal delays. Each transducer is equipped with a latency buffer that compensates for any discrepancies in signal arrival times, thereby preventing desynchronization due to variations in wireless communication.

In one exemplary embodiments, the system may be utilized for cognitive therapies, where the plurality of effectors 806 deliver synchronized stimulation to specific areas of the patient's body that correlate with targeted brain regions. The feedback device monitors brainwave activity in real time, and the control unit adjusts the pulse frequency and intensity to optimize cognitive function.

In one another exemplary embodiment, the system 800 is applied in neuro-rehabilitation for patients recovering from stroke, wherein the plurality of effectors 806 are positioned on the patient's limbs to stimulate motor control centers in the brain. The feedback processing unit 816 analyzes muscle response data and modulates the waveform and intensity of the pulses to promote neural recovery and plasticity.

In one another exemplary embodiment, the system is adapted to dynamically adjust stimulation parameters based on real-time feedback, thereby improving the precision and effectiveness of the therapy. The system's synchronization capabilities ensure that all stimulation pulses are delivered in a coordinated manner, maintaining neural coherence, which is critical for brain-related therapies.

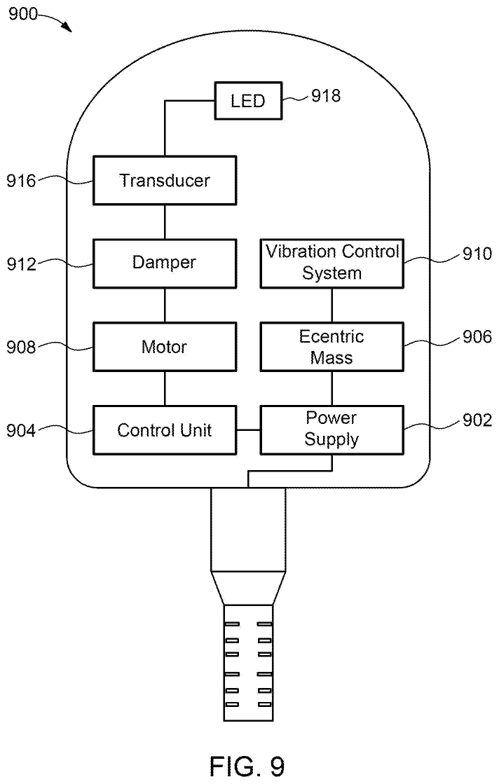

Referring to FIG. 9 illustrates a detailed schematic diagram of effectors according to an embodiment of the present disclosure. The effector 900 is designed to generate kinetic stimulation when triggered and forms a key component in the system for brain therapy, enabling controlled stimulation for maintaining neural synchronization. The effector 900 comprises a power supply unit 902 that provides electrical power to drive the various components of the effector 900. The control unit 904 is operatively connected to the power supply unit and manages the operation of the effector 900 based on commands received from a central control module, which may be controlled either by the patient or by the practitioner.

In one exemplary embodiment of the present disclosure, the effector 900 is equipped with an eccentric mass 906 that is mechanically linked to a motor 908. When the motor is activated by the control unit 904, it rotates the eccentric mass 906, generating vibratory tapping motion that serves as the kinetic stimulation applied to the patient's body.

Further, intensity, frequency, and duration of this vibration are modulated by a vibration control system 910, which allows the effector to deliver stimulation tailored to the specific needs of the patient. Additionally, a damper 912 is included to absorb excess vibrations and prevent overstimulation, ensuring that the kinetic pulses remain within therapeutic thresholds while providing a smoother and more comfortable experience for the patient.

In one another exemplary embodiment of the present disclosure, the effector 900 further includes a transducer 916, which is adapted to convert electrical signals from the control unit into mechanical vibrations, supplementing the vibratory tapping output of the motor. This transducer 916 is dynamically controlled based on real-time inputs received from the central control module, ensuring that the stimulation delivered by the effector 900 is synchronized with other effectors in the system.

In addition, an LED 918 is incorporated into the effector to provide visual feedback on the operational status of the device, indicating whether the effector is active, idle, or experiencing a fault.

In one another exemplary embodiment of the present disclosure, the control unit 904 within the effector 900 controls the operation of the motor 908, the damper 912, the vibration control system 910, and the transducer 916, ensuring that each module functions cohesively to deliver the intended therapeutic stimulation. The control unit adjusts the effector's output based on real-time feedback from the patient, processed through the central control module, which is equipped with a feedback loop to continuously optimize therapy.

In one another exemplary embodiment of the present disclosure, when used on the body of a patient, the effector 900 is attached to a specific treatment area, such as the limbs, scalp, or other regions corresponding to targeted neural pathways. Once activated, the motor 908 rotates the eccentric mass 906, generating kinetic stimulation that are transmitted through the body to engage the nervous system. The vibration control system 910 modulates these vibrations to align with the therapy's desired outcomes, adjusting intensity and frequency based on the patient's response.

Furthermore, the damper 912 ensures that excess vibrations are dissipated, preventing discomfort or overstimulation. Simultaneously, the transducer 916 is configured to convert electrical feedback signals into mechanical energy, further enhancing the precision and effectiveness of the treatment.

In one another exemplary embodiment of the present disclosure, the effector 900 can be manufactured in a variety of types, shapes, and materials to suit different therapeutic applications. Specifically, the effectors may take the form of circular, rectangular, or custom-shaped pads that conform to the contours of the patient's body, depending on the targeted area of stimulation. For example, circular effectors may be used for scalp stimulation, while rectangular pads might be employed on larger muscle groups.

In one another exemplary embodiment of the present disclosure, the materials used for the outer surface of the effectors may include soft, biocompatible silicone, medical-grade plastics, or flexible polymers designed to provide comfort while ensuring effective contact with the patient's skin. In addition to silicone and polymer-based designs, more rigid materials may be used for effectors intended to target specific pressure points or deeper tissue areas, where higher-intensity kinetic stimulation is required.

In one another exemplary embodiment of the present disclosure, the interior components, including the motor and transducer, are typically housed in protective casings that are resistant to moisture and designed to withstand repeated use. For high-durability applications, the internal components may be shielded by lightweight aluminum or other metals, ensuring longevity while maintaining a compact form factor suitable for extended therapeutic sessions.

In one another exemplary embodiment of the present disclosure, the effector's combination of the eccentric mass 906 and motor 908 generates the kinetic stimulation critical for engaging the patient's neural pathways. The vibration control system 910 and damper 912 ensure that the stimulation remains within the desired therapeutic range, preventing overstimulation while providing targeted therapy.

Further, the inclusion of the transducer 916 refines the system by converting electrical signals into mechanical vibrations, enhancing synchronization with other effectors and improving therapeutic precision. In this regard, the visual feedback provided by the LED 918 offers an additional layer of operational transparency, enabling the practitioner or patient to monitor the status of the effector in real time.

In one another exemplary embodiment, the effector is placed on the patient's limb, delivering synchronized kinetic stimulation that promotes motor recovery by reactivating neural circuits associated with motor control. The vibration control system adjusts the frequency and intensity based on feedback from the patient, ensuring that the stimulation is neither too weak to be effective nor too strong to cause discomfort.

In another exemplary embodiment, the effector is applied to the scalp in a cognitive enhancement therapy, delivering low-frequency vibrations to stimulate brain regions associated with learning and memory. The damper 912 smooths out excessive vibrations to ensure patient comfort during longer sessions, while the transducer 916 ensures that stimulation remains synchronized across multiple effectors.

In one another exemplary embodiment of the present disclosure, the control unit 904 operates in conjunction with the central control module to adjust the effector's performance in real-time based on patient-specific feedback, enhancing the efficacy and safety of the therapy. Further, the transducer 916 supports the motor in delivering synchronized mechanical stimulation, ensuring that all effectors operate in unison to maintain neural coherence throughout the therapy session. This synchronization is critical for optimizing therapeutic outcomes in brain therapy applications.

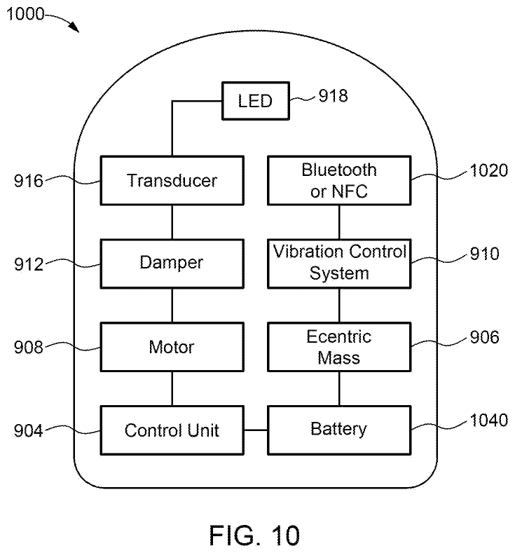

Referring to FIG. 10, a detailed schematic diagram of an effector 1000 with wireless connectivity is illustrated in accordance with another embodiment of the present disclosure. This embodiment, while maintaining the kinetic stimulation functionality as described in FIG. 9, introduces key additional features, including wireless connectivity and an integrated power source, thereby enhancing the flexibility and usability of the effector 1000 during brain therapy.

In particular, the effector 1000 includes a battery 1040, typically a rechargeable battery, which provides continuous power to the effector 1000, enabling extended operation without the need for external power connections. This battery 1040 ensures that the effector 1000 can function independently for prolonged periods, allowing for uninterrupted therapy sessions. The battery 1040 is specifically designed to support the energy demands of the motor, vibration control system, and other components integrated within the effector 1000.

In one exemplary embodiment of the present disclosure, the wireless connection module 1020 enables the effector 1000 to communicate wirelessly with the central control module of the brain therapy system. The wireless connection module 1020 facilitates the transmission of control signals to and from the effector 1000, allowing real-time adjustments to be made to the intensity, frequency, and timing of the stimulation pulses based on feedback received either from the patient or input provided by the practitioner.

In one another exemplary embodiment of the present disclosure, the wireless module also enables seamless synchronization between multiple effectors, ensuring that all devices operate in harmony without the need for physical connections, thus offering greater flexibility in the placement of effectors on the patient's body.

Further, the remaining modules, including the control unit, eccentric mass, motor, vibration control system, and damper, operate similarly to those described in FIG. 9. These components collectively generate kinetic stimulation in the form of vibrations, with the vibration control system regulating the parameters of the stimulation to ensure synchronization and alignment with the therapeutic goals of the session. The damper mitigates excess vibrations to prevent overstimulation and improve patient comfort.

In one another exemplary embodiment of the present disclosure, the incorporation of the wireless connection module 1020 and inbuilt power source 1010 provides critical technical advancements, enabling the effector 1000 to function wirelessly, thus removing the need for cumbersome wiring and enhancing the portability and ease of use of the system. In addition, the wireless communication not only facilitates more flexible and patient-friendly therapy but also ensures precise coordination between effectors, crucial for maintaining neural coherence during brain therapy.

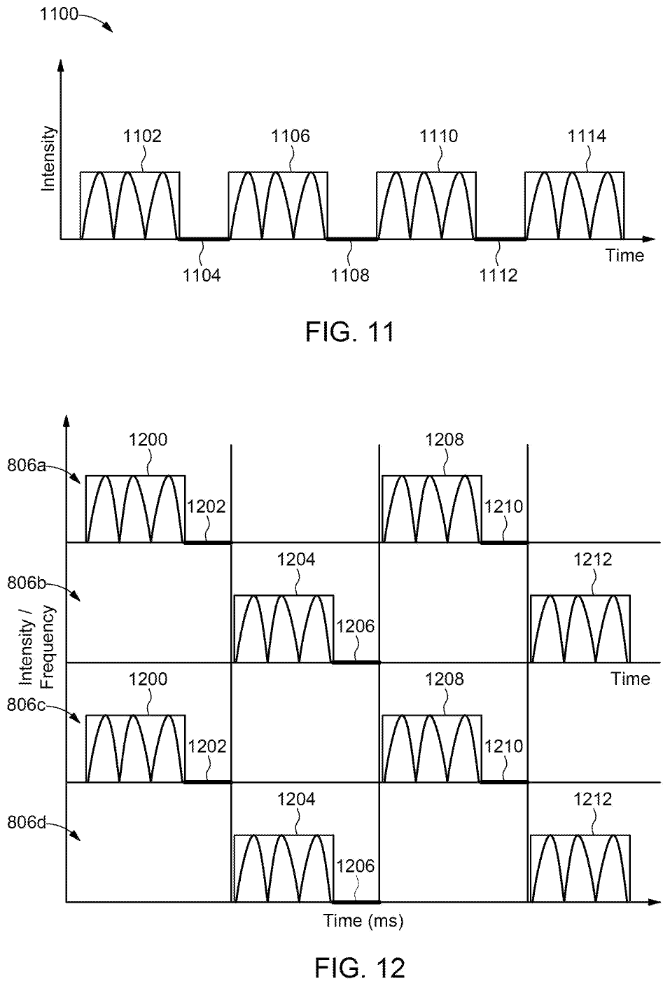

Referring to FIG. 11, a pulse pattern used during brain therapy is illustrated in accordance with an embodiment of the present disclosure. The neuromodulation system 1100 is configured to generate wave signals of a single frequency, forming a wave train over a predetermined time duration 1102. These wave trains are separated by delay interval 1104, with each wave train being superimposed over a carrier frequency to achieve targeted modulation of neural pathways during therapy. The system 1100 utilizes uniform sinusoidal waves generated by the control unit 802 at various time intervals, specifically 1102, 1106, 1110, and 1114, with corresponding delay interval 1104, 1108, and 1112. The frequency, intensity, and delay interval are dynamically controlled by the control unit 802 based on the specific stimulation requirements of the patient.

In one exemplary embodiment of the present disclosure, the delay intervals, such as those represented by 1104, 1108, and 1112, illustrate the timing gaps between the activation or firing of the effector pairs during a therapy session. The delay intervals between the activation of the first pair of effectors (e.g., 806a and 806c) and the second pair of effectors (e.g., 806b and 806d) are programmable and may be set by the system based on the specific therapeutic requirements set by the practitioner and physiological conditions of the patient.

In one another embodiment, the delay intervals may be automatically configured by the system based on real-time patient data received from a plurality of sensors positioned on the patient's body. These sensors are configured to measure various physiological parameters, such as neural responses, muscle tension, heart rate, and skin conductivity. The sensor data is processed by the feedback processing unit, which automatically adjusts the delay intervals between the activation of the effector pairs. This real-time adjustment ensures that the therapy is dynamically responsive to the patient's ongoing physiological conditions, enabling a more personalized and adaptive therapeutic approach.

Additionally, in another exemplary embodiment, the system may operate without any delay intervals between the activations of the first and second pairs of effectors. In this embodiment, the stimulation pulses are delivered continuously or in immediate succession, without any timing gap between the activation of the effector pairs.

Moreover, the duration of the active phase during which the effectors are delivering stimulation pulses is also programmable. The practitioner can adjust the duration of the active stimulation period according to the therapeutic requirements, ranging from brief pulses to prolonged stimulation intervals. In an alternative embodiment, the system may automatically adjust the duration of the active phase based on real-time patient data processed by the feedback system. This automatic adjustment ensures that the therapy remains responsive to the patient's condition and that the duration of stimulation is continually optimized to maximize therapeutic benefits.

Further, the system's capability to dynamically adjust both the delay intervals and the duration of the active stimulation phase, either manually by the practitioner or automatically based on patient data allows the system to deliver highly tailored therapy. The flexibility of the system to operate with either programmable delay intervals or no delay intervals further enhances its adaptability to various therapeutic applications, ensuring that the system can be customized for a wide range of neurological conditions and therapeutic goals.

In one exemplary embodiment of the present disclosure, the sinusoidal waves serve to stimulate neural pathways by being delivered at precise intervals. Further, the superimposition over a carrier frequency allows for accurate modulation targeting specific regions of the brain. The customizable nature of the pulse pattern enables the system to adapt to a variety of therapeutic needs, including adjustments in frequency, intensity, and delay interval depending on the patient's feedback and therapeutic goals.

In one another exemplary embodiment of the present disclosure, the control unit 802 plays an essential role in maintaining this precision by dynamically adjusting the pulse parameters in real-time, ensuring that the therapy remains responsive to the patient's neurological condition and feedback.

In one another exemplary embodiment of the present disclosure, the pulse pattern generated by the neuromodulation system 1100 is used in cognitive enhancement therapy. The sinusoidal waves are superimposed over a low-frequency carrier to target brain regions associated with memory and learning, with the control unit 802 adjusting the delay interval and frequency in response to the patient's brainwave activity. This dynamic adjustment ensures optimal stimulation for cognitive function improvement.

In another exemplary embodiment of the present embodiment, the pulse pattern is employed in neuro-rehabilitation therapy for patients recovering from traumatic brain injury. The system generates high-frequency sinusoidal waves with short intervals to stimulate motor control centers in the brain. The delay interval are adjusted in real-time during the therapy session to synchronize with the patient's neural responses, promoting recovery and reactivation of motor pathways.

In one exemplary embodiment of the present disclosure, the pulse pattern can also be adapted for use in pain management therapy, where low-frequency waves with extended intervals are employed to modulate pain pathways in the brain. The control unit 802 customizes the wave parameters to match the patient's pain profile, ensuring effective pain reduction while maintaining patient comfort throughout extended therapy sessions.

Referring to FIG. 12, a detailed illustration of the synchronized delivery of two frequencies for brain therapy is shown in accordance with an embodiment of the present disclosure. The system synchronizes the operation of four effectors by first triggering two effectors at one frequency, followed by a delayed activation of a second pair of effectors at a synchronized frequency. The effectors depicted as 320, 340, 360, and 380 in FIG. 3B correspond to the effector pairs 806a, 806b, 806c, and 806d shown in FIG. 8.

In one exemplary embodiment of the present disclosure, upon activation, the first pair of effectors 806a and 806c are triggered to generate kinetic stimulation for a specified time interval 1200, followed by a delay interval 1202. Subsequently, the second pair of effectors 806b and 806d are triggered for time interval 1204, followed by a corresponding delay interval 1206. This cyclical process continues, with the first pair of effectors 806a and 806c being re-triggered at interval 1208 and deactivated at 1210, while the second pair of effectors 806b and 806d are reactivated at interval 1212. The alternating stimulation delivered by these synchronized effectors ensures continuous and coordinated neuromodulation throughout the therapy session.

In one exemplary embodiment of the present disclosure, the system supports a range of frequencies to accommodate different therapeutic applications. Lower frequencies may be used for relaxation and pain modulation, while higher frequencies can be employed for cognitive enhancement or neuro-rehabilitation.

In one another exemplary embodiment of the present disclosure, by alternating the activation of effector pairs with precise delay intervals, the system prevents overstimulation and ensures that kinetic pulses are consistently delivered across the patient's body. This controlled timing sequence allows for more effective neuromodulation, enhancing therapeutic outcomes while maintaining patient safety. The system's ability to modulate frequencies based on the therapeutic need further optimizes treatment for specific neurological conditions.

In one exemplary embodiment of the present disclosure, the system is employed for cognitive enhancement therapy, with the first pair of effectors 806a and 806c delivering low-frequency stimulation to brain regions associated with attention and focus, while the second pair 806b and 806d provides higher-frequency stimulation to memory-related regions. The alternating sequence ensures effective stimulation of multiple brain areas without overlap, optimizing cognitive function.

For example, as shown in FIG. 12, synchronization is achieved through the central control unit, which generates timing signals that are distributed to the transducers. In one scenario, the first pair of transducers 806a and 806c may be triggered simultaneously for a specific time interval 1200, followed by a delay interval 1202. After this delay, the second pair of transducers 806b and 806d is activated for a subsequent interval 1204, creating an alternating pattern of synchronized stimulation. The control unit uses software-controlled algorithms to calculate and adjust these intervals dynamically, ensuring that the transducers remain synchronized throughout the therapy session.

In wireless configurations, timestamped communication signals are transmitted to each transducer, ensuring that all transducers are activated at the correct moment, despite any potential delays in signal propagation. Latency compensation mechanisms are further employed to adjust for any discrepancies in the timing of signals, ensuring that the transducers remain in sync even in wireless environments.

In systems that rely on real-time feedback, synchronization is maintained through continuous monitoring of the patient's physiological responses. Sensors provide data on the timing and intensity of the stimulation pulses, and based on this feedback, the control unit dynamically adjusts the timing of subsequent pulses to maintain synchronization.

In one exemplary embodiment of the present disclosure, synchronization in the system is achieved through feedback from sensors attached to the patient's body, which continuously monitor the timing and intensity of kinetic stimulation and adjust the operation of the transducers accordingly. The transducers receive timestamped wireless signals from the central control unit, and based on these signals, each transducer adjusts its internal timing to ensure synchronization with the other transducers. The central control unit calculates latency adjustments to account for any signal delays, thereby ensuring that all transducers remain synchronized despite variations in signal propagation time. The system also employs phase-locking techniques to maintain a consistent phase relationship between the transducers, ensuring that they operate in unison without drift or desynchronization.

Additionally, the system dynamically updates therapy parameters and recalculates timing intervals in response to real-time patient feedback, thereby ensuring that the therapy remains synchronized and responsive to the patient's evolving condition throughout the session.

In one another exemplary embodiment of the present disclosure, stimulating a first limb located on a first side of a median plane and a first side of a transverse plane, while simultaneously stimulating a second limb located on a second, opposite side of the median plane and the second, opposite side of the transverse plane. Subsequently, a third limb located on the second side of the median plane and the first side of the transverse plane is stimulated, while a fourth limb located on the first side of the median plane and the second side of the transverse plane is also stimulated.

synchronized within a range of less than 200 milliseconds to ensure therapeutic outcomes by maintaining neural synchronization across the body. The synchronization is critical to achieving a coherent and harmonized neural response, which is necessary for effective neuromodulation during brain therapy.

In one another exemplary embodiment of the present disclosure, the system dynamically adjusts the stimulation timing between the first, second, third, and fourth limbs within a range of 50 to 200 milliseconds, depending on patient-specific data collected in real-time during the therapy session. This real-time adjustment allows the system to respond to the patient's unique neurological condition and to optimize the synchronization of the stimulation pulses to maintain a consistent therapeutic effect.

In addition, the synchronized timing ensures that stimulation pulses delivered to the limbs are coordinated, preventing desynchronization or neural interference, which could reduce the efficacy of the therapy. The ability to adjust the frequency and timing based on real-time feedback allows the therapy to be highly individualized, enhancing patient outcomes and providing more effective brain therapy.

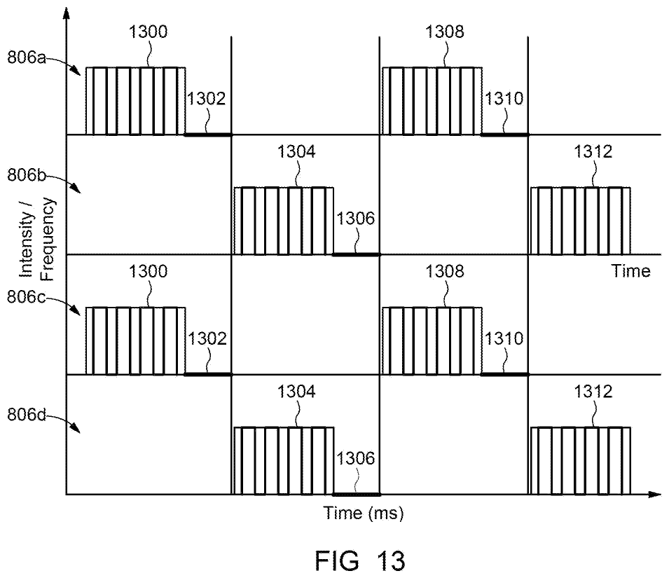

Referring to FIG. 13 illustrates the synchronized delivery of two frequencies for brain therapy according to another embodiment of the present disclosure. This embodiment is similar to the system described in FIG. 12, with the primary difference being the introduction of a different waveform, such as a square wave, for stimulation. The timing diagram remains consistent with FIG. 12, involving synchronized activation of two pairs of effectors. The system alternates between two pairs of effectors while utilizing varied waveform characteristics to achieve optimized neuromodulation based on the patient's therapeutic needs.

Upon activation, the first pair of effectors 806a and 806c are triggered to deliver kinetic stimulation for a defined time interval 1300, followed by a delay interval 1302. Subsequently, the second pair of effectors 806b and 806d are triggered during time interval 1304, followed by a delay interval 1306. This cyclical process repeats, with the first pair of effectors 806a and 806c being re-triggered at interval 1308 and deactivated at 1310, while the second pair of effectors 806b and 806d are triggered again at interval 1312. The synchronized delivery of pulses ensures continuous and coordinated stimulation across different regions of the body, following the same temporal patterns as described in FIG. 12.

However, the distinguishing feature of FIG. 13 is the use of a square waveform for the stimulation pulses, in contrast to the sinusoidal waveforms depicted in FIG. 12. Square waveforms deliver sharp, consistent pulses of stimulation, which are particularly beneficial for certain types of neuromodulation. In addition to square waves, the system is capable of generating other waveforms, including triangular, sawtooth, or custom waveforms, depending on the specific therapeutic application.

In particular, the choice of waveform is determined by the type of therapy required, with square waves offering high precision and rapid pulse delivery that can be advantageous for stimulating motor neurons or for therapies that require a more direct neural response. In this regard, the use of different waveforms offers significant flexibility in treatment options. Square waves, with their abrupt transitions, are particularly effective for stimulating motor neurons and enhancing neuroplasticity, making them ideal for neuro-rehabilitation and motor function therapies. Triangular or sawtooth waveforms may be more suitable for therapies requiring smoother transitions, such as cognitive enhancement or relaxation therapies. This capability to tailor the waveform ensures that the therapy is not only adaptable but also optimized for each patient's unique neurological profile.

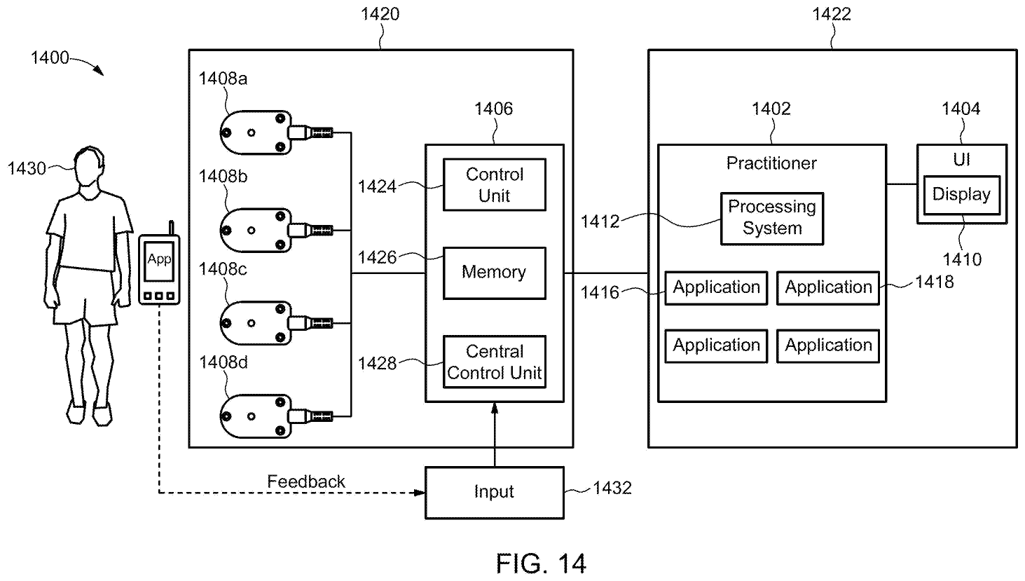

Referring to FIG. 14 illustrates a block diagram of a system 1400 for delivering therapy or treating patients, in accordance with an embodiment of the present disclosure. The system 1400 is designed to facilitate the administration of brain therapy through precise control of kinetic stimulation, delivered via a series of effectors. The system 1400 includes a therapy stimulator 1420, which is operated by a control device 1402 by receiving inputs from a practitioner 1422. The control device 1402 is equipped with a processing system 1412 and a software application comprising a set of instructions 1416 that enable the practitioner 1422 to update various therapy-related parameters. The control device 1402 is further provided with a user interface 1404, which is displayed on a display device 1410, allowing the practitioner to monitor the ongoing therapy session in real time. The control device 1402 is communicatively linked to the therapy stimulator 1420, either via a wired or wireless connection, enabling the practitioner to control and adjust therapy settings as necessary.

In one exemplary embodiment of the present disclosure, the therapy stimulator 1420 includes a processing unit 1406 configured to manage the internal components of the system. In this regard, the processing unit 1406 contains a control unit 1424, which regulates key therapy parameters, such as intensity, frequency, and waveform. Additionally, a memory 1426 is provided for storing instructions and therapy protocols, while a central control unit 1428 is configured to control the overall operation of the stimulator 1420. The central control unit 1428 processes instructions received from the control device 1402 and manages the delivery of kinetic stimulation through the effectors 1408a, 1408b, 1408c, and 1408d. The central control unit 1428 also receives external feedback from the patient 1430 via a feedback mechanism 1432, allowing for real-time adjustment of therapy parameters in response to the patient's condition.

Further, the instructions 1416 provided by the practitioner through the control device 1402 are processed by the central control unit 1428, which generates corresponding signals to control the operation of the control unit 1406. Particularly, these signals are then transmitted to the effectors 1408a, 1408b, 1408c, and 1408d, which deliver synchronized kinetic stimulation to the patient. The synchronization of the effectors ensures that therapy is delivered consistently and uniformly across the patient's body, maintaining neural coherence. The therapy parameters are continuously updated based on feedback received from the patient 1430 via a user interface 1432 on a user device held by the patient, allowing for dynamic adjustments during the session to optimize treatment outcomes. The user device is communicatively coupled with the stimulator 1420 to transmit the feedback for updating the therapy parameters.

In one exemplary embodiment of the present disclosure. the central control unit 1428 is configured to process patient feedback and dynamically adjusts therapy parameters, ensuring that the therapy remains responsive and adaptive to the patient's evolving needs. The real-time adjustment capability is essential for maintaining neural synchronization, which is vital for achieving therapeutic outcomes in brain therapy. Additionally, the control device 1402 with its user interface 1404 enables the practitioner to monitor and adjust the therapy parameters in real time.

In one exemplary embodiment, the practitioner uses the control device 1402 to monitor a patient's responses and adjust therapy settings accordingly. The central control unit 1428 modulates the timing and intensity of the kinetic pulses delivered by the effectors 1408a, 1408b, 1408c, and 1408d, promoting the recovery of motor function by stimulating targeted neural pathways. The synchronized operation of the effectors 1408a, 1408b, 1408c, and 1408d maintains neural coherence.

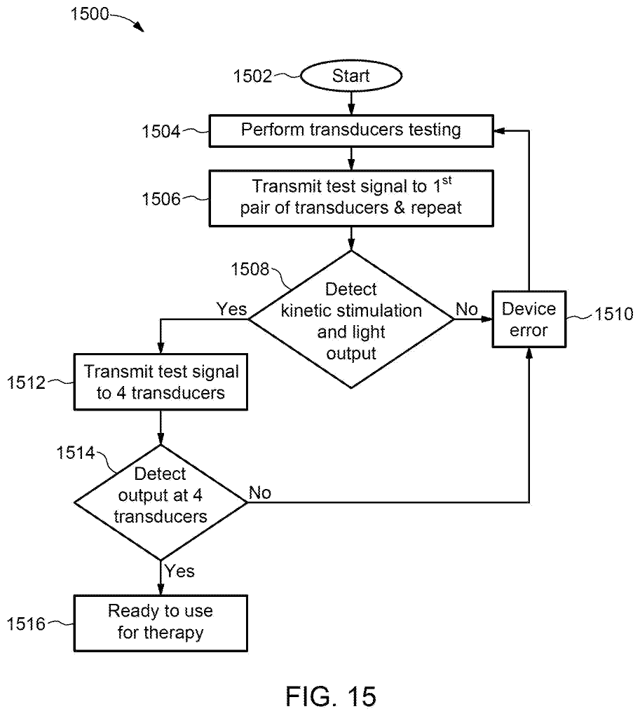

Referring to FIG. 15 illustrates a flowchart 1500 outlining a method for testing the functionality of a stimulator system prior to initiating a therapy session. The process commences at step 1502, where the stimulator device undergoes pre-therapy testing to ensure operational integrity and to detect any potential device errors or false alarms. At step 1504, each transducer (e.g. effector) within the system is individually tested to verify whether it is functioning correctly or exhibiting a fault.

At step 1506, a test signal is transmitted to the first pair of transducers to assess their output, after which kinetic stimulation is detected at step 1508 using various sensors, such as accelerometers to measure vibration intensity, pressure sensors to detect mechanical output, and optical sensors for monitoring light emission, if applicable. These sensors ensure comprehensive monitoring of the transducers' kinetic output and any auxiliary signals, verifying proper functionality.

In one exemplary embodiment of the present disclosure, when no signal is detected at step 1508, the system identifies a malfunction in the transducers, leading to a device error being displayed on the user interface at step 1510, alerting the practitioner of the issue and preventing the device from being used for therapy. Conversely, in case the signals are properly detected, the process advances to step 1512, where a second test signal is transmitted to all four transducers simultaneously. At step 1514, the output of all four transducers is evaluated to ensure synchronized functionality across the system. In case any of the outputs are not detected, the process reverts to step 1510, indicating a device error.

Further, in case the outputs are detected successfully, the system proceeds to step 1516, confirming that the stimulator device is fully operational, synchronized, and ready for therapeutic use. Thus, this testing protocol is critical in preventing device malfunctions during therapy, thereby safeguarding the patient and maintaining the consistency of therapeutic delivery. By testing both individual transducers and the system as a whole, kinetic stimulation is delivered as intended and that synchronization across the transducers is maintained.

In one another exemplary embodiment of the present disclosure, the integration of accelerometers, pressure sensors, and optical sensors for monitoring various aspects of transducer output provides a robust testing mechanism that enhances the accuracy of device diagnostics. This ensures that any malfunction, whether in kinetic stimulation or light output, is promptly detected and addressed before therapy begins.

In one another exemplary embodiment of the present disclosure, the method is employed in a neuro-rehabilitation therapy system, where precise and consistent kinetic stimulation is essential for engaging motor pathways in the brain. The pre-therapy testing ensures that the transducers are fully operational and capable of delivering synchronized stimulation, thus preventing interruptions in the treatment and supporting effective motor recovery.

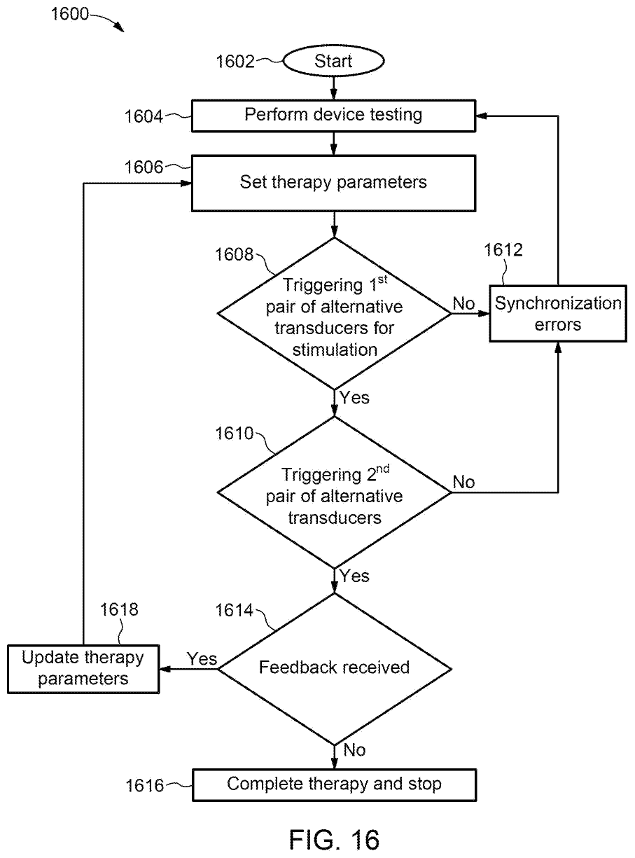

Referring to FIG. 16 illustrates a flowchart 1600 depicting an exemplary method for synchronizing a stimulator system during a therapeutic session. The process begins at step 1602, where the stimulator device is prepared for use in a therapy session. At step 1604, a pre-testing procedure is conducted, as described in FIG. 15, to ensure that the device is fully functional and that all transducers are operating correctly. Once pre-testing is complete, therapy parameters are set at step 1606, based on inputs either from the patient or the practitioner. Inputs from the patient are gathered via various sensors attached to the patient's body, which monitor physiological parameters such as heart rate, muscle tension, or skin conductivity. These inputs are processed to automatically adjust therapy parameters to suit the patient's condition. Alternatively, the practitioner/patient may manually input therapy parameters, using clinical observations to tailor the treatment.

In one exemplary embodiment of the present disclosure, at step 1608, the first pair of transducers is triggered to deliver kinetic stimulation, for example, to the right hand and left foot. The system monitors the synchronization of the transducers during this process. In case the triggering is not synchronized, a synchronization error is displayed on the user interface (UI of practitioner/patient) at step 1612, and the system reverts to step 1604 to re-initiate device testing. However, in case the transducers are synchronized, the process proceeds to step 1610, where the second pair of transducers is triggered to stimulate other areas, such as the left hand and right foot. This alternating stimulation process continues in a cyclical manner for the prescribed duration of the therapy session.

During the session, at step 1614, feedback may be received from the patient, such as real-time information regarding comfort or the effectiveness of the therapy. Based on this feedback, the system proceeds to step 1618, where it requests updates to the therapy parameters. The process then returns to step 1606 to adjust and update the therapy settings, ensuring that the treatment remains optimized in response to the patient's evolving condition. If no feedback is received, the system moves to step 1616, completing the therapy session once the designated time has elapsed, at which point the device powers down or enters standby mode, signaling the conclusion of the treatment.

Embodiments of the disclosure are susceptible to being used for various purposes, including, though not limited to, enabling users to improve brain function.

The benefit of disclosed methods and systems is unlimited since everyone has a brain and would like it to work to the best of its capability. People with PTSD, ADHD, ADD and TBI, amateur and professional athletes as well as highly skilled professionals such as doctors, surgeons, entertainment performers, accountants, attorneys, politicians and professors could benefit from disclosed methods and systems. Finally, some animals may also benefit from disclosed systems and methods.

Modifications to embodiments of the disclosure described in the foregoing are possible without departing from the scope of the disclosure as defined by the accompanying claims. Expressions such as “including”, “comprising”, “incorporating”, “consisting of”, “have”, “is” used to describe and claim disclosed features are intended to be construed in a non-exclusive manner, namely allowing for items, components or elements not explicitly described also to be present. Reference to the singular is also to be construed to relate to the plural.

We claim:

1. A method for providing brain therapy to maintain neural synchronization through timed and controlled stimulation pulses, the method comprising:
stimulating a first limb of a body located on a first side of a median plane and a first side of a transverse plane, while stimulating a second limb located on a second, opposite side of the median plane and a second, opposite side of the transverse plane;
subsequently stimulating a third limb located on a second side of the median plane and the first side of the transverse plane, while stimulating a fourth limb located on the first side of the median plane and the second side of the transverse plane,
wherein the median plane is defined as a vertical plane that divides the body into left and right halves, and the transverse plane is defined as a horizontal plane that divides the body into upper and lower parts;
dynamically adjusting synchronization of the stimulation pulses across the limbs based on timing signals generated by a software-controlled algorithm; and
calibrating the synchronization of the stimulation pulses across the limbs by monitoring and recalculating a distribution of neural activity based on real-time patient data.

2. The method of claim 1, wherein the synchronization timing of the stimulation pulses is dynamically adjusted within a range of 100 to 200 milliseconds, based on timing signals generated by the software-controlled algorithm.

3. The method of claim 1, further comprising automatically adjusting a frequency and intensity of the stimulation pulses based on real-time system feedback from the software-controlled algorithm, while maintaining synchronization within a range of less than 200 milliseconds.

4. The method of claim 1, wherein the synchronization of the stimulation pulses occurs with a delay of less than 150 milliseconds, based on clock-based synchronization signals.

5. The method of claim 1, further comprising varying stimulation intensity between the first and second limbs, and the third and fourth limbs, with intensity modulation occurring in synchronization with the pulse timing based on the software-controlled algorithm.

6. The method of claim 1, wherein the stimulation pulses are delivered in predetermined waveform patterns selected from sinusoidal, square, or triangular, with a waveform type being dynamically selected by a control system based on system-specific feedback and patient-specific neural response profiles.

7. The method of claim 1, wherein the stimulation pulses are synchronized by distributing clock signals to a control unit and effectors and wherein the synchronization of the stimulation pulses is further adjusted based on real-time measurements of electrical impedance from electrodes attached to the body of a patient.

8. The method of claim 1, further comprising modulating intensity and frequency of the stimulation pulses in response to detected changes in heart rate of a patient.

9. The method of claim 1, further comprising:
dynamically adjusting, via a control system, a waveform type, intensity, and frequency of the stimulation pulses based on feedback signal received from a plurality of sensors sensing physiological parameters of a patient; and
dynamically adjusting the synchronization and intensity of the stimulation pulses based on a feedback signal measured by a plurality of sensors.

10. A system for providing brain therapy to maintain neural synchronization through timed and controlled stimulation pulses, the system comprising:
a plurality of transducers configured to be positioned on a first limb located on a first side of a median plane and a first side of a transverse plane, and a second limb located on a second, opposite side of the median plane and a second, opposite side of the transverse plane, as well as a third limb located on a second side of the median plane and the first side of the transverse plane, and a fourth limb located on the first side of the median plane and the second side of the transverse plane;
a control unit operatively connected to the plurality of transducers, the control unit configured to generate and deliver stimulation pulses to the transducers;
a timing circuit or software-controlled algorithm in the control unit, configured to dynamically adjust the synchronization of the stimulation pulses across the limbs by generating timing signals; and
a feedback processing unit operatively connected to sensors configured to monitor physiological data of a patient in real-time, the feedback processing unit configured to recalibrate and adjust the synchronization of the stimulation pulses based on a distribution of neural activity and patient-specific physiological data;
wherein the control unit is configured to adjust synchronization of the stimulation pulses.

11. The system of claim 10, wherein the control unit is further configured to adjust synchronization timing of the stimulation pulses within a range of 100 to 200 milliseconds, based on timing signals generated by the software-controlled algorithm.

12. The system of claim 10, wherein the control unit is further configured to automatically adjust frequency and intensity of the stimulation pulses based on real-time feedback processed by the feedback processing unit, while maintaining synchronization within a range of less than 200 milliseconds.

13. The system of claim 10, wherein the synchronization of the stimulation pulses occurs with a delay of less than 150 milliseconds, based on clock-based synchronization signals generated by the control unit.

14. The system of claim 10, wherein the control unit is further configured to vary stimulation intensity between the first and second limbs, and the third and fourth limbs, wherein intensity modulation occurs in synchronization with the pulse timing and is controlled by the software-controlled algorithm.

15. The system of claim 10, wherein the stimulation pulses are delivered in predetermined waveform patterns selected from sinusoidal, square, or triangular, and a waveform type is dynamically selected by the control unit based on feedback from patient-specific neural response profiles.

16. The system of claim 10, wherein the synchronization of the stimulation pulses is controlled by distributing clock signals to the control unit and the plurality of transducers, and further adjusted based on real-time measurements of electrical impedance from electrodes attached to a body of the patient.

17. The system of claim 10, wherein the control unit is further configured to modulate intensity and frequency of the stimulation pulses in response to detected changes in heart rate of patient.

18. The system of claim 10, wherein the control unit is further configured to dynamically adjust waveform type, intensity, and frequency of the stimulation pulses based on feedback signals received from a plurality of sensors configured to sense physiological parameters of the patient.

19. The system of claim 10, wherein the control unit is further configured to dynamically adjust the synchronization and intensity of the stimulation pulses based on real-time feedback signals measured by a plurality of sensors monitoring physiological data of the patient.

20. A non-transitory computer-readable medium storing instructions that, when executed by one or more processors, cause a system to perform a method for providing brain therapy to maintain neural synchronization through timed and controlled stimulation pulses, the method comprising:
stimulating a first limb of a body located on a first side of a median plane and a first side of a transverse plane, while stimulating a second limb located on a second, opposite side of the median plane and a second, opposite side of the transverse plane;
subsequently stimulating a third limb located on a second side of the median plane and the first side of the transverse plane, while stimulating a fourth limb located on the first side of the median plane and the second side of the transverse plane;
wherein the median plane is defined as a vertical plane that divides the body into left and right halves, and the transverse plane is defined as a horizontal plane that divides the body into upper and lower parts;
dynamically adjusting synchronization of the stimulation pulses across the limbs based on timing signals generated by a software-controlled algorithm;
calibrating the synchronization of the stimulation pulses across the limbs by monitoring and recalculating a distribution of neural activity based on real-time data collected from patient-specific physiological parameters; and
generating control signals to deliver said stimulation pulses to a plurality of transducers operatively connected to the limbs, said control signals ensuring that the synchronization of stimulation pulses is maintained within specified timing intervals to optimize therapeutic outcomes.
